(19) United States
(12) Patent Application Publication
(10) Pub. No.: US 2016/0287601 A1
(43) Pub. Date: Oct. 6, 2016

(54) ENHANCED TREATMENT REGIMENS USING PI3K INHIBITORS

(71) Applicant: MILLENNIUM PHARMACEUTICALS, INC., Cambridge, MA (US)

(72) Inventors: Fabian ZOHREN, Newton, MA (US); Chirag PATEL, Boston, MA (US)

(21) Appl. No.: 15/026,352

(22) PCT Filed: Oct. 2, 2014
(86) PCT No.: PCT/US2014/058926

§ 371 Date: Mar. 31, 2016

Related U.S. Application Data
(60) Provisional application No. 61/886,623, filed on Oct. 3, 2013.
(60) Provisional application No. 62/054,879, filed on Sep. 24, 2014.

(51) Int. Cl. A61K 31/5377; A61K 31/565; A61K 45/06

(52) U.S. Cl. CPC A61K 31/5377; A61K 45/06; A61K 31/565

(57) ABSTRACT

The present invention provides for methods and pharmaceutical compositions comprising inhibitors of phosphatidylinositol 3-kinases (PI3Ks). In some aspects, the invention provides for treatment regimens resulting in enhanced treatment efficacy and better tolerability. In other aspects, the invention provides for methods of treatment and treatment regimens comprising combination of a phosphatidylinositol 3-kinase (PI3Ks) inhibitor and an estrogen receptor antagonist.

### CROSS-REFERENCE TO RELATED APPLICATIONS

This application claims the benefit of U.S. Provisional Patent Application No. 61/886,623, filed on Oct. 3, 2013, and U.S. Provisional Patent Application No. 62/054,879, filed on Sep. 24, 2014, each of which is incorporated herein by reference in its entirety.

### BACKGROUND OF THE INVENTION

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases (Gaestel et al. Current Medicinal Chemistry (2007) 14:2214-2234).

The phosphoinositide 3-kinases (PI3Ks) signaling pathway is one of the most highly mutated systems in human cancers. PI3K signaling is also a key factor in many other diseases in humans. PI3K signaling is involved in many disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

The phosphoinositide 3-kinases (PI3Ks) are members of a unique and conserved family of intracellular lipid kinases that phosphorylate the 3′—OH group on phosphatidylinositols or phosphoinositides. The PI3K family comprises 15 kinases with distinct substrate specificities, expression patterns, and modes of regulation (Katso et al., 2001). The class I PI3Ks (p110α, p110β, p110δ, and p110γ) are typically activated by tyrosine kinases or G-protein coupled receptors to generate phosphatidylinositol-3,4,5-trisphosphate (PIP₃), which engages downstream effectors such as those in the Akt/PDK1 pathway, mTOR, the Tec family kinases, and the Rho family GTPases. The class II and III PI3-Ks play a key role in intracellular trafficking through the synthesis of PI(3)P and PI(3,4)P2. The PIKKs are protein kinases that control cell growth (mTORC1) or monitor genomic integrity (ATM, ATR, DNA-PK, and hSmg-1). The production of PIP₃ initiates potent growth and survival signals. In some epithelial cancers the PI3K pathway is activated by direct genetic mutation. As PI3K signaling pathway plays a pivotal role in cell proliferation and differentiation, inhibition of this pathway has been shown to be beneficial in hyperproliferative diseases.

The alpha (a) isoform of PI3K (PI3Ks) has been implicated, for example, in a variety of human cancers. Angiogenesis has been shown to selectively require the a isoform of PI3K in the control of endothelial cell migration. (Graupera et al, Nature 2008; 453; 662-6). Mutations in the gene coding for PI3Kα or mutations which lead to upregulation of PI3Kα are believed to occur in many human cancers such as lung, stomach, endometrial, ovarian, bladder, breast, colon, brain and skin cancers. Often, mutations in the gene coding for PI3Kα are point mutations clustered within several hotspots in helical and kinase domains, such as E542K, E545K, and H1047R. Many of these mutations have been shown to be oncogenic gain-of-function mutations. Because of the high rate of PI3K mutations, targeting of this pathway may provide valuable therapeutic opportunities. While other PI3K isoforms such as PI3Kγ or PI3Kδ are expressed primarily in hematopoietic cells, PI3Kα, along with PI3Kβ, is expressed constitutively.

Downstream mediators of the PI3K signal transduction pathway include Akt and mammalian target of rapamycin (mTOR). Akt possesses a pleckstrin homology (PH) domain that bind PIP3, leading to Akt kinase activation. Akt phosphorylates many substrates and is a central downstream effector of PI3K for diverse cellular responses. Full activation of Akt typically requires phosphorylation of T308 in the activation loop and S473 in a hydrophobic motif. One important function of Akt is to augment the activity of mTOR, through phosphorylation of TSC2 and other mechanisms.

Dysregulation of signaling pathways mediated by many other kinases is a key factor in the development of human diseases. Aberrant or excessive protein kinase activity or expression has been observed in many disease states including benign and malignant proliferative diseases, disorders such as allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

As such, kinases particularly protein kinases such as mTor and Akt, as well as lipid kinases such as PI3Ks are prime targets for drug development.

At the same time, tolerability and safety are important considerations in structuring courses of treatment for many diseases. For example, treatments using therapeutic agents that result in severe adverse events may become ineffective clinically due to insufficient patient compliance or because an effective therapeutic dose cannot be safely administered to the patient.

Estrogen receptor antagonists are anticancer agents used for treating cancers wherein estrogen has been implicated in cell proliferation. Such cancers include breast cancer and uterine cancer. However, many estrogen receptor antagonists are not particularly effective particularly after relapse or in advanced cancer. Thus, there still remains a considerable need for alternative regimens and/or treatments for wide variety of cancers.

### SUMMARY OF THE INVENTION

The present invention addresses the need for an acceptable safety and efficacy window for PI3Kα inhibition by providing treatment regimens utilizing methods and compositions of the PI3Kα inhibitor described below. Treatments that result in a higher effective concentration of the drug being present in the blood stream for a longer period of time may provide better therapeutic efficacy. In addition, a combination treatment involving PI3Kα and estrogen receptor antagonists can improve the therapeutic efficacy against a variety of cancers.

Accordingly in one aspect, the invention provides a pharmaceutical regimen for the treatment of a disorder mediated by PI3-kinase α (PI3Kα) comprising administering intermittently for at least one week a PI3Kα inhibitor to a human subject in need thereof. In some and each of the embodiments described below, the PI3Kα inhibitor can be (6-(2-aminobenzo[d]oxazol-5-yl)imidazo[1,2-a]pyridin-3-yl)(morpholino)methanone (i.e., compound A in the Figures described below), and has the following structure:

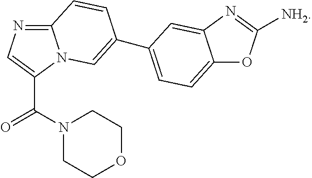 [SMILES: Nc1nc2cc(-c3ccc4ncc(C(=O)N5CCOCC5)n4c3)ccc2o1]

In some embodiments, the regimen allows for a weekly maximum tolerated dose (MTD) higher than 600 mg of the PI3Kα inhibitor. In some embodiments, the regimen allows for a weekly maximum tolerated dose (MTD) higher than 1050 mg of the PI3Kα inhibitor. In other embodiments, the regimen allows for a weekly maximum tolerated dose (MTD) higher than 1400 mg of the PI3Kα inhibitor. In some embodiments, the regimen allows for a weekly maximum tolerated dose (MTD) higher than 300 mg, higher than 900 mg, higher than 1800 mg, higher than 2700 mg, or higher than 3600 mg of the PI3Kα inhibitor.

In some embodiments, the regimen allows for a weekly maximum tolerated dose (MTD) from about 600 mg to about 3000 mg of the PI3Kα inhibitor. In some embodiments, the regimen allows for a weekly maximum tolerated dose (MTD) from about 300 mg to about 3600 mg of the PI3Kα inhibitor. In other embodiments, the regimen allows for a weekly maximum tolerated dose (MTD) from about 900 mg to about 3000 mg of the PI3Kα inhibitor. In yet other embodiments, the regimen allows for a weekly maximum tolerated dose (MTD) from about 1200 mg to about 3000 mg of the PI3Kα inhibitor. In still other embodiments, the regimen allows for a weekly maximum tolerated dose (MTD) from about 1200 mg to about 2700 mg of the PI3Kα inhibitor. In further embodiments, the regimen allows for a weekly maximum tolerated dose (MTD) from about 1800 mg to about 2700 mg of the PI3Kα inhibitor.

In some embodiments, the regimen allows for a weekly maximum tolerated dose (MTD) of about 2700 mg of said PI3Kα inhibitor. In other embodiments, the regimen allows for a weekly maximum tolerated dose (MTD) of about 3000 mg of said PI3Kα inhibitor. In some embodiments, the regimen allows for a weekly maximum tolerated dose (MTD) of about 2700 mg of said PI3Kα inhibitor. In other embodiments, the regimen allows for a weekly maximum tolerated dose (MTD) of about 3600 mg of said PI3Kα inhibitor.

In another aspect, the invention provides a pharmaceutical regimen for the treatment of a disorder comprising administering intermittently for at least one week a pharmaceutical composition comprising a PI3-kinase α (PI3Kα) inhibitor to a human subject in need thereof. In some embodiments, the PI3Kα inhibitor is (6-(2-aminobenzo[d]oxazol-5-yl)imidazo[1,2-a]pyridin-3-yl)(morpholino)methanone.

In some embodiments, the intermittent regimen yields an area under the curve (AUC) that is at least comparable to that obtained by administering the pharmaceutical composition once daily when the total weekly dosage administered according to said regimen is comparable or less than that of the daily administration of 300 mg (i.e., 2100 mg/wk). In other embodiments, the intermittent regimen yields an AUC that is at least comparable to that obtained by administering the pharmaceutical composition once daily when the total weekly dosage administered according to said regimen is comparable or less than that of said daily administration of 200 mg (e.g., 1400 mg/wk). In yet other embodiments, the intermittent regimen yields an AUC that is at least comparable to that obtained by administering the pharmaceutical composition once daily when the total weekly dosage administered according to said regimen is comparable or less than that of said daily administration of 150 mg (e.g., 1050 mg/wk).

In some embodiments, the invention provides a pharmaceutical regimen comprising at least one cycle in which the PI3Kα inhibitor is administered for at least one day followed by an intermission in which the PI3Kα inhibitor is not administered for at least one day. In other words, the cycle contains at least one drug-free or non-dosing day. In some embodiments, the pharmaceutical regimen comprises administering the PI3Kα inhibitor for 2, 3, 4, 5, 6, or 7 consecutive days, followed by an intermission in which the PI3Kα inhibitor is not administered for at least 1, 2, 3, 4, 5, or 6 days. In some embodiments, the PI3Kα inhibitor is administered to the human subject three days a week. In some embodiments, the PI3Kα inhibitor is administered to the human subject every other day.

In some embodiments, the pharmaceutical regimen comprises administering the PI3Kα inhibitor to the human subject on consecutive days during the week and followed by an intermission of at least 1, 2, 3, 4, 5, or 6 days. In further embodiments, the regimen comprises at least one 7-day cycle in which the PI3Kα inhibitor is administered for 3 consecutive days followed by an intermission of 4 consecutive days.

In some embodiments, the pharmaceutical regimen comprises administering the PI3Kα inhibitor to the human subject on alternate days during the week and involving an intermission in between the days PI3Kα inhibitor is administered. In further embodiments, the regimen comprises administering the PI3Kα inhibitor at least 3 times on alternative days within a 7-day cycle.

In some embodiments, the pharmaceutical regimen comprises administering the PI3Kα inhibitor once a day (QD) in each of the days that the PI3Kα inhibitor is administered to the human subject. In other embodiments, the pharmaceutical regimen comprises administering the PI3Kα inhibitor twice a day (BID) in each of the days that the PI3Kα inhibitor is administered to the human subject. Such dosing may apply to any form of intermittent regimen disclosed herein. For example, the intermittent regimen involves administering the PI3Kα inhibitor QD to the subject on 3 dosing days within a 7-day cycle at a dosage that ranges from 300 to 3600 mg/wk or from, 1800 to 3600 mg/wk (at about 1800 mg/wk or at about 2700 mg/wk or at about 3600 mg/wk). As another example, the intermittent regimen involves administering the PI3Kα inhibitor BID to the subject on 3 dosing days within a 7-day cycle at a dosage that ranges from 1800 to 3600 mg/wk (at about 1800 mg/wk or at about 2700 mg/wk or at about 3600 mg/wk).

In some embodiments, the pharmaceutical regimen achieves an area under the curve (AUC) greater than 45 μg*h/mL (e.g., greater than 48.5 μg*h/mL or greater than 50 μg*h/mL or greater than 60 μg*h/mL or greater than 75 μg*h/mL) in the subject over one dosing day. In some embodiments, the pharmaceutical regimen achieves an area under the curve (AUC) greater than 100 μg*h/mL in the subject over a week. In other embodiments, the regimen achieves an area under the curve (AUC) greater than 150 μg*h/mL in the subject over a week. In yet other embodiments, the regimen achieves an area under the curve (AUC) greater than 200 μg*h/mL in the subject over a week.

In some embodiments, the intermittent dosing regimen allows for a longer treatment duration (e.g., 1, 2, 3, 4, 5, 6, 7, 8, 9, 10 weeks or longer) compared to the daily dosing regimen using comparable subjects and dosages of the PI3Kα inhibitor (e.g., 600-3600 mg/wk, for example, at about 1800 mg/wk or at about 2700 mg/wk or at about 3600 mg/wk).

In some embodiments, the pharmaceutical regimen does not result in significant liver enzyme elevation in the human subject. Elevation of liver enzyme can be determined by measuring the level of serum alanine aminotransferase (ALT) or serum aspartate aminotransferase (AST). In some embodiments, the pharmaceutical regimen does not result in an increase in the liver enzyme level to more than 2.5 fold of the upper limit of normal (ULN) after a week. In some embodiments, the pharmaceutical regimen does not result in an increase in the liver enzyme level to more than 5.0 fold of the upper limit of normal (ULN) after a week.

In some embodiments pharmaceutical regimen comprises administering an additional therapeutic agent to the subject. In some embodiments, the additional therapeutic agent is an anticancer agent. In further embodiments, the additional therapeutic agent is selected from one or more of paclitaxel, fulvestrant, exemestane, gemcitabine, erlotinib, gefitinib, afatinib, nintedanib, dacomitinib, bevacizumab, pemetrexed, motesanib, crizotinib, ipilimumab, ramucirumab, custirsen, and onartuzumab. In other embodiments, the addition therapeutic agent is fulvestrant. The additional therapeutic agent can be administered with the PI3Kα inhibitor concomitantly or independently.

In one aspect, the invention provides a method of treating a neoplastic condition in a subject in need thereof comprising administering a therapeutically effective amount of a combination of a PI3K-kinase α (PI3Kα) inhibitor and an estrogen receptor antagonist. Where desired, the PI3Kα inhibitor is administered simultaneously with an estrogen receptor antagonist. In some embodiments, the PI3Kα inhibitor is administered intermittently. In some embodiments, the PI3Kα inhibitor is administered intermittently and is a compound of the following formula:

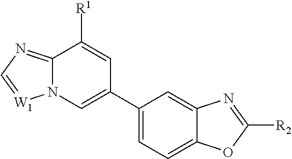

or a pharmaceutically acceptable salt thereof, wherein
W¹ is CR³;
R¹ is hydrogen;
R² hydrogen, alkyl, heteroalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, sulfonamido, halo, cyano, hydroxy, nitro, phosphate, urea, carbonate, or NR′R″ wherein R′ and R″ are taken together with nitrogen to form a cyclic moiety; and
R³ is amido of formula —C(O)N(R)₂ or —NHC(O)R, wherein R is selected from the group consisting of hydrogen, alkyl, cycloalkyl, aryl, heteroaryl and heteroalicyclic; or wherein the (R)₂ groups taken together with the nitrogen to which it is attached form a 4-, 5-, 6-, or 7-membered ring.

In some embodiments, the neoplastic condition is a cancer selected from the group consisting of non-small cell lung cancer, small cell lung cancer, head and neck squamous cell carcinoma, pancreatic cancer, breast cancer, ovarian cancer, renal cell carcinoma, prostate cancer, neuroendocrine cancer, gastric cancer, bladder cancer, colon cancer and endometrial cancer. In some embodiments, the neoplastic condition is breast cancer. In some embodiments, the breast cancer is positive for estrogen receptor expression as determined by a hormone receptor assay. In some embodiments, the breast cancer is positive for the expression of estrogen receptor, progesterone receptor and human epidermal growth factor receptor 2 as determined by a hormone receptor assay.

In another but related aspect, the invention provides for a pharmaceutical regimen for the treatment of a disorder mediated by PI3-kinase α (PI3Kα), wherein the regimen comprises administration of at least one estrogen receptor antagonist and at least one PI3Kα inhibitor to a human subject in need thereof. Where desired, the PI3Kα inhibitor is administered simultaneously with an estrogen receptor antagonist. In some embodiments, the PI3Kα inhibitor is administered intermittently. In another aspect, the invention provides for a pharmaceutical regimen for the treatment of a disorder mediated by PI3-kinase α (PI3Kα), wherein the regimen comprises administration of at least one estrogen receptor antagonist and at least one PI3Kα inhibitor to a human subject in need thereof, wherein the PI3Kα inhibitor is administered intermittently, and wherein the PI3Kα inhibitor is a compound of the following formula:

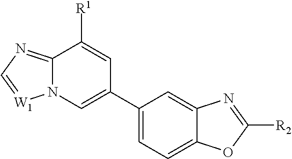

or a pharmaceutically acceptable salt thereof, wherein
W¹ is CR³;
R¹ is hydrogen;
R² is hydrogen, alkyl, heteroalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, sulfonamido, halo, cyano, hydroxy, nitro, phosphate, urea, carbonate, or NR′R″ wherein R′ and R″ are taken together with nitrogen to form a cyclic moiety; and
R³ is amido of formula —C(O)N(R)₂ or —NHC(O)R, wherein R is selected from the group consisting of hydrogen, alkyl, cycloalkyl, aryl, heteroaryl and heteroalicyclic; or wherein the (R)₂ groups taken together with the nitrogen to which it is attached form a 4-, 5-, 6-, or 7-membered ring.

In some embodiments, the disorder is a cancer selected from the group consisting of non-small cell lung cancer, small cell lung cancer, head and neck squamous cell carcinoma, pancreatic cancer, breast cancer, ovarian cancer, renal cell carcinoma, prostate cancer, neuroendocrine cancer, gastric cancer, bladder cancer, colon cancer and endometrial cancer. In some embodiments, the disorder is breast cancer. In some embodiments, the breast cancer is positive for estrogen receptor expression as determined by a hormone receptor assay. In some embodiments, the breast cancer is positive for the expression of estrogen receptor, progesterone receptor and human epidermal growth factor receptor 2 as determined by a hormone receptor assay.

In some embodiments, the PI3Kα inhibitor is administered on consecutive days in a 7-day cycle followed by an intermission. In some embodiments, the PI3Kα inhibitor is administered on alternate days in a 7-day cycle followed by an intermission.

In some embodiments, the PI3Kα inhibitor is administered for at least 1 day, followed by an intermission in which the PI3Kα inhibitor is not administered for at least 1 day for at least one cycle. In some embodiments, the PI3Kα inhibitor is administered for 1, 2, 3, 4, 5, 6 or 7 consecutive days followed by an intermission in which the PI3Kα inhibitor is not administered for at least 1 day. In some embodiments, the PI3Kα inhibitor is administered for 1, 2, 3, 4, 5, 6 or 7 consecutive days followed by an intermission of at least 1, 2, 3, 4, 5, or 6 consecutive days. In some embodiments, the PI3Kα inhibitor is administered for 2, 3, 4, 5, 6 or 7 consecutive days followed by an intermission of at least 1, 2, 3, 4, 5, or 6 consecutive days. In some embodiments, the PI3Kα inhibitor is administered for 7, 8, 9, 10, 11, 12, 13, or 14 consecutive days, followed by an intermission where the PI3Kα inhibitor is not administered for at least 1, 2, 3, 4, 5, 6, or 7 days. In other embodiments, the PI3Kα inhibitor is administered for 2, 3, 4, 5, 6 or 7 consecutive days followed by an intermission in which the PI3Kα inhibitor is not administered for at least 3, 4, or 5 consecutive days.

In some embodiments, the PI3Kα inhibitor is administered on consecutive days in a 7-day cycle followed by an intermission. In other embodiments, the PI3Kα inhibitor is administered for 3 consecutive days followed by an intermission of 4 consecutive days for at least one 7-day cycle. In some embodiments, the PI3Kα inhibitor is administered for 3 consecutive days of a 7-day cycle followed by an intermission of at least one day. In some embodiments, the PI3Kα inhibitor is administered for 3 consecutive days followed by an intermission of 4 consecutive days per 7-day cycle. In yet other embodiments, the PI3Kα inhibitor is administered for 4 consecutive days followed by an intermission of 3 consecutive days for at least one 7-day cycle. In yet other embodiments, the PI3Kα inhibitor is administered for 5 consecutive days followed by an intermission of 2 consecutive days for at least one 7-day cycle. In yet other embodiments, the PI3Kα inhibitor is administered for 6 consecutive days followed by an intermission of 1 day for at least one 7-day cycle.

In some embodiments, the PI3Kα inhibitor is administered for at least 1 day for at least one 7-day cycle. In some embodiments, the PI3Kα inhibitor is administered for at least 2 days for at least one 7-day cycle. In some embodiments, the PI3Kα inhibitor is administered for at least 3 days for at least one 7-day cycle. In some embodiments, the PI3Kα inhibitor is administered for at least 4 days for at least one 7-day cycle. In some embodiments, the PI3Kα inhibitor is administered for at least 5 days for at least one 7-day cycle. In some embodiments, the PI3Kα inhibitor is administered for at least 6 days for at least one 7-day cycle.

In some embodiments, the PI3Kα inhibitor is administered every other day. In some embodiments, the PI3Kα inhibitor is administered on three non-consecutive days in a 7-day cycle. In some embodiments, the PI3Kα inhibitor is administered on alternate days in a 7-day cycle. In some embodiments, the PI3Kα inhibitor is administered on alternate days in a 7-day cycle followed by an intermission. In some embodiments, the PI3Kα inhibitor is administered at least 3 times on alternate days within a 7-day cycle. In some embodiments, the PI3Kα inhibitor is administered at least 4 times on alternate days within a 7-day cycle.

In some embodiments, the PI3Kα inhibitor is administered to a subject within a range of about an amount of 300 mg to about 3600 mg of the PI3Kα inhibitor in a 7-day cycle. In some embodiments, the PI3Kα inhibitor is administered to a subject within a range of about an amount of 900 mg to about 3600 mg of the PI3Kα inhibitor in a 7-day cycle. In some embodiments, the amount of PI3Kα inhibitor administered in a 7-day cycle is about 300 mg. In some embodiments, the amount of PI3Kα inhibitor administered in a 7-day cycle is about 900 mg. In some embodiments, the amount of PI3Kα inhibitor administered in a 7-day cycle is about 1800 mg. In some embodiments, the amount of PI3Kα inhibitor administered in a 7-day cycle is about 2700 mg. In some embodiments, the amount of PI3Kα inhibitor administered in a 7-day cycle is about 3600 mg.

In some embodiments, a dose of the PI3Kα inhibitor is about 100 to about 1200 mg per a single administration. In some embodiments, a dose of the PI3Kα inhibitor is about 300 to about 1200 mg per a single administration. In some embodiments a dose of the PI3Kα inhibitor is about 100 mg, about 300 mg, about 600 mg, or about 900 mg per a single administration. In some embodiments a dose of the PI3Kα inhibitor is about 300 mg, about 600 mg, or about 900 mg per a single administration. In some embodiments a dose of the PI3Kα inhibitor is about 100 mg, 300 mg, about 600 mg, about 900 mg, or about 1200 mg per a single administration. In some embodiments, a dose of the PI3Kα inhibitor is about 300 mg, about 600 mg, about 900 mg, or about 1200 mg per a single administration. In some embodiments, a dose of the PI3Kα inhibitor is about 100 mg per a single administration. In some embodiments, a dose of the PI3Kα inhibitor is about 300 mg per a single administration. In some embodiments, a dose of the PI3Kα inhibitor is about 600 mg per a single administration. In some embodiments, a dose of the PI3Kα inhibitor is about 900 mg per a single administration. In some embodiments, a dose of the PI3Kα inhibitor is about 1200 mg per a single administration.

In some embodiments, the estrogen receptor antagonist is a selective estrogen receptor modulator. In some embodiments, the estrogen receptor antagonist is a selective estrogen receptor downregulator. In some embodiments, the estrogen receptor antagonist is raloxifene, tamoxifen, toremifene, droloxifene, idoxifene, arzoxifene, or fulvestrant. In some embodiments, the estrogen receptor antagonist is fulvestrant.

In some embodiments, R³ is amido of formula —C(O)N(R)₂ wherein the (R)₂ groups taken together with the nitrogen to which they are attached form a 4-, 5-, 6-, or 7-membered ring. In a further embodiment, R³ is amido of formula —C(O)N(R)₂ wherein the (R)₂ groups taken together with the nitrogen to which they are attached form a 6-membered ring. In a further embodiment, R³ is amido of formula —C(O)N(R)₂ wherein the (R)₂ groups taken together with the nitrogen to which they are attached form a morpholinyl ring. In some embodiments, R² is amino. In a further embodiment, R² is NH₂.

In some embodiments, R³ is amido of formula —C(O)N(R)₂ wherein the (R)₂ groups taken together with the nitrogen to which they are attached form a 4-, 5-, 6-, or 7-membered ring and R² is amino. In a further embodiment, R³ is amido of formula —C(O)N(R)₂ wherein the (R)₂ groups taken together with the nitrogen to which they are attached form a 6-membered ring and R² is amino. In a further embodiment, R³ is amido of formula —C(O)N(R)₂ wherein the (R)₂ groups taken together with the nitrogen to which they are attached form a morpholinyl ring and R² is NH₂.

In some embodiments, the PI3Kα inhibitor is a compound with the following structure:

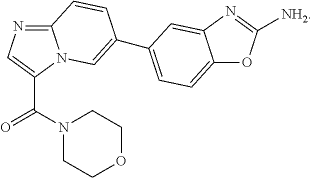 [SMILES: Nc1nc2cc(-c3ccc4ncc(C(=O)N5CCOCC5)n4c3)ccc2o1]

or a pharmaceutically acceptable salt thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the tumor reduction efficacy observed upon administration of compound A according to various regimens.

FIG. 2 shows the tumor reduction efficacy observed upon administration of compound A according to various regimens that delivered the same amount of compound A per week.

FIG. 3 shows the linear relationship between exposure (AUC) and efficacy (tumor growth inhibition TGI).

FIG. 4 shows pharmacokinetic data (i.e. Cₘₐₓ/dose, MRT_{inf}, and AUC_{inf}/dose) for compound A administered at various dose levels.

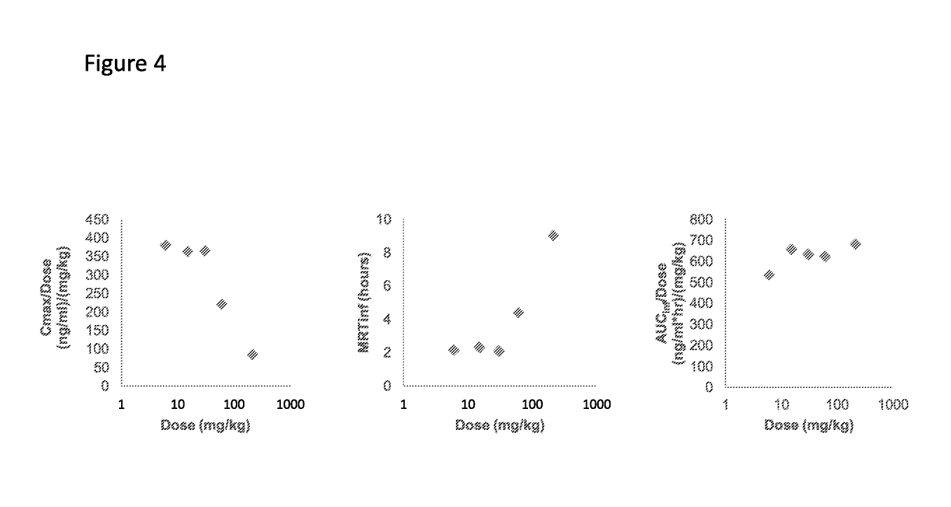

FIG. 5 shows correlation between PK parameters with efficacy.

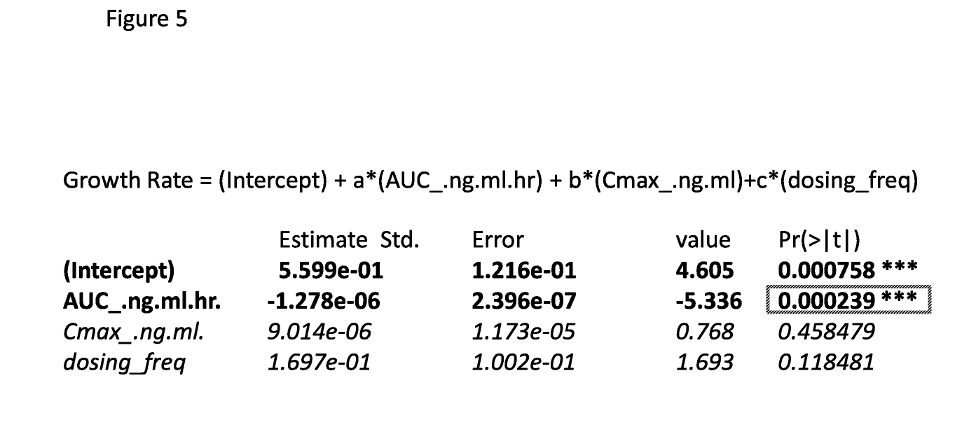

FIG. 6 shows efficacy of compound A as a single agent, efficacy of fulvestrant as a single agent, and the combination efficacy of both drugs dosed concurrently over a 21 day treatment period.

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. Unless stated otherwise, the present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Treatment”, “treating”, “palliating” and “ameliorating”, as used herein, are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, the term “neoplastic condition” or “neoplastic disorder” refers to the presence of cells possessing abnormal growth characteristics, such as uncontrolled proliferation, immortality, metastatic potential, rapid growth and proliferation rate, perturbed oncogenic signaling, and certain characteristic morphological features. This includes but is not limited to the growth of: (1) benign or malignant cells (e.g., tumor cells) that correlates with overexpression of a tyrosine or serine/threonine kinase; (2) benign or malignant cells (e.g., tumor cells) that correlates with abnormally high level of tyrosine or serine/threonine kinase activity or lipid kinase activity. Exemplary tyrosine kinases implicated in a neoplastic condition include but are not limited to receptor tyrosine kinases such as epidermal growth factor receptors (EGF receptor), platelet derived growth factor (PDGF) receptors, and cytosolic tyrosine kinases such as src and abl kinase. Non-limiting serine/threonine kinases implicated in neoplastic condition include but are not limited to raf, mek, mTor, and akt. Exemplary lipid kinases include but are not limited to PI3 kinases such as PI3Kα, PI3Kβ, PI3Kδ, and PI3Kγ.

The term “effective amount” or “therapeutically effective amount” refers to that amount of an inhibitor described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

A “synergistically effective” therapeutic amount or “synergistically effective” amount of an agent or therapy is an amount which, when combined with an effective or sub-therapeutic amount of another agent or therapy, produces a greater effect than when either of the two agents are used alone. In some embodiments, a synergistically effective therapeutic amount of an agent or therapy produces a greater effect when used in combination than the additive effects of each of the two agents or therapies when used alone. The term “greater effect” encompasses not only a reduction in symptoms of the disorder to be treated, but also an improved side effect profile, improved tolerability, improved patient compliance, improved efficacy, or any other improved clinical outcome.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “agonist” or “activator” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor, or an undesired immune response as manifested in autoimmune disease.

The phrase “PI3Kα inhibitor” refers to a PI3Kα inhibitor that interacts with and reduces activity of PI3Kα kinase. For example, the PI3Kα inhibitor can be (6-(2-aminobenzo[d]oxazol-5-yl)imidazo[1,2-a]pyridin-3-yl)(morpholino)methanone and its pharmaceutically acceptable salts, prodrugs, or radioactive isomers. As used herein, compound A is (6-(2-aminobenzo[d]oxazol-5-yl)imidazo[1,2-a]pyridin-3-yl)(morpholino)methanone, and has the following structure:

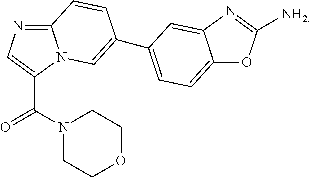 [SMILES: Nc1nc2cc(-c3ccc4ncc(C(=O)N5CCOCC5)n4c3)ccc2o1]

An “anti-neoplastic”, “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The terms “co-administration,” “administered in combination with,” and their grammatical equivalents, encompass administration of two or more agents to an animal so that both agents and/or their metabolites are present in the animal at the same time. Co-administration includes simultaneous administration in separate compositions, administration at different times in separate compositions, or administration in a composition in which both agents are present. Co-administered agents may be in the same formulation. Co-administered agents may also be in different formulations.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Signal transduction” is a process during which stimulatory or inhibitory signals are transmitted into and within a cell to elicit an intracellular response. A modulator of a signal transduction pathway refers to a compound that modulates the activity of one or more cellular proteins mapped to the same specific signal transduction pathway. A modulator may augment (agonist) or suppress (antagonist) the activity of a signaling molecule.

“Subject” refers to an animal, such as a mammal, for example a human. The methods described herein can be useful in both human therapeutics, pre-clinical, and veterinary applications. In some embodiments, the subject is a mammal, and in some embodiments, the subject is human.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The following abbreviations and terms have the indicated meanings throughout: PI3K=Phosphoinositide-3-kinase; PI=phosphatidylinositol.

Unless otherwise stated, the connections of compound name moieties are at the rightmost recited moiety. That is, the substituent name starts with a terminal moiety, continues with any linking moieties, and ends with the linking moiety. For example, heteroarylthio C₁₋₄ alkyl has a heteroaryl group connected through a thio sulfur to a C₁₋₄ alkyl radical that connects to the chemical species bearing the substituent. This condition does not apply where a formula such as, for example “-L-C₁₋₁₀ alkyl —C₃₋₈cycloalkyl” is represented. In such case, the terminal group is a C₃₋₈cycloalkyl group attached to a linking C₁₋₁₀ alkyl moiety which is attached to an element L, which is itself connected to the chemical species bearing the substituent.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “halo” or “halogen” refers to fluoro, chloro, bromo, or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl, and the like.

“Acyl” refers to the groups (alkyl)-C(O)—, (aryl)-C(O)—, (heteroaryl)-C(O)—, (heteroalkyl)-C(O)—, and (heterocycloalkyl)-C(O)—, wherein the group is attached to the parent structure through the carbonyl functionality. In some embodiments, it is a C₁-C₁₀ acyl radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e., C₃-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. In some embodiments, it is a C₃-C₈ cycloalkyl radical. In some embodiments, it is a C₃-C₅ cycloalkyl radical. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

As used herein, the term “heteroatom” or “ring heteroatom” is meant to include oxygen (O), nitrogen (N), sulfur (S), phosphorus (P), and silicon (Si).

“Heteroalkyl” includes optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given, e.g., C₁-C₄ heteroalkyl which refers to the chain length in total, which in this example is 1 to 4 atoms long. For example, a —CH₂OCH₂CH₃ radical is referred to as a “C₄” heteroalkyl, which includes the heteroatom center in the atom chain length description. Connection to the rest of the molecule may be through either a heteroatom or a carbon in the heteroalkyl chain. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “haloalkenyl” refers to an alkenyl group substituted with one or more halo groups.

Unless otherwise specified, the term “cycloalkenyl” refers to a cyclic aliphatic 3 to 8 membered ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl, and the like.

“Alkynyl” refers to a straight or branched hydrocarbon chain radical group consisting solely of carbon and hydrogen atoms, containing at least one triple bond, having from two to ten carbon atoms (i.e., C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C₂-C₅ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl and each of these moieties may be optionally substituted as defined herein.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. In some embodiments it is a C₁-C₄ amido or amide radical, which includes the amide carbonyl in the total number of carbons in the radical. The R^{2′} of —N(R)₂ of the amide may optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroaryl” or, alternatively, “heteroaromatic” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteraryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The terms “aryl-alkyl”, “arylalkyl” and “aralkyl” are used to describe a group wherein the alkyl chain can be branched or straight chain forming a linking portion with the terminal aryl, as defined above, of the aryl-alkyl moiety. Examples of aryl-alkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl, and 10-phenyldecyl. Either portion of the moiety is unsubstituted or substituted.

The terms “heteroarylalkyl”, “heteroarylalkyl”, “heteroaryl-alkyl”, “heteroaryl-alkyl”, “hetaralkyl” and “heteroaralkyl” are used to describe a group wherein the alkyl chain can be branched or straight chain forming a linking portion of the heteroaralkyl moiety with the terminal heteroaryl portion, as defined above, for example 3-furylmethyl, thenyl, furfuryl, and the like. Either portion of the moiety is unsubstituted or substituted.

The term “heterocyclyl” refers to a four-, five-, six-, or seven-membered ring containing one, two, three or four heteroatoms independently selected from nitrogen, oxygen and sulfur. The four-membered ring has zero double bonds, the five-membered ring has zero to two double bonds, and the six- and seven-membered rings have zero to three double bonds. The term “heterocyclyl” also includes bicyclic groups in which the heterocyclyl ring is fused to another monocyclic heterocyclyl group, or a four- to seven-membered aromatic or nonaromatic carbocyclic ring. The heterocyclyl group can be attached to the parent molecular moiety through any carbon atom or nitrogen atom in the group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heterocycloalkyl group may consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C₃-C₁₀ heterocycloalkyl. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

The terms “heterocyclylalkyl”, “heterocyclyl-alkyl”, “hetcyclylalkyl”, and “hetcyclylalkyl” are used to describe a group wherein the alkyl chain can be branched or straight chain forming a linking portion of the heterocyclylalkyl moiety with the terminal heterocyclyl portion, as defined above, for example 3-piperidinylmethyl and the like. The term “heterocycloalkylene” refers to the divalent derivative of heterocycloalkyl.

The term “alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl, is an alkyl group which encompasses both straight and branched chain alkyls of from 1 to 4 carbon atoms.

The term “alkylthio” includes both branched and straight chain alkyl groups attached to a linking sulfur atom, for example methylthio and the like.

The term “oxo” refers to an oxygen that is double bonded to a carbon atom. One in the art understands that an “oxo” requires a second bond from the atom to which the oxo is attached. Accordingly, it is understood that oxo cannot be substituted onto an aryl or heteroaryl ring, unless it forms part of the aromatic system as a tautomer.

“Sulfonamidyl” or “sulfonamido” refers to a —S(═O)₂—NR′R′ radical, where each R′ is selected independently from the group consisting of hydrogen, alkyl, cycloalkyl, aryl, heteroaryl (bonded through a ring carbon) and heteroalicyclic (bonded through a ring carbon). The R′ groups in —NR′R′ of the —S(═O)₂—NR′R′ radical may be taken together with the nitrogen to which it is attached to form a 4-, 5-, 6-, or 7-membered ring. A sulfonamido group is optionally substituted by one or more of the substituents described for alkyl, cycloalkyl, aryl, heteroaryl respectively.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. Compounds may be shown without a definitive stereochemistry at certain positions. The present invention includes all stereoisomers of the disclosed compounds and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The present invention includes all manner of rotamers and conformationally restricted states of an inhibitor of the invention.

Substituents for alkyl, heteroalkyl, cycloalkyl, heterocycloalkyl monovalent and divalent derivative radicals (including those groups often referred to as alkylene, alkenyl, heteroalkylene, alkynyl, cycloalkyl, and heterocycloalkyl) can be one or more of a variety of groups selected from, but not limited to: alkyl, heteroalkyl, alkenyl, alkynyl, cycloalkyl, heterocycloalkyl, aryl, arylalkyl, heteroaryl, heteroarylalkyl, —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When an inhibitor of the invention includes more than one R group, for example, each of the R groups is independently selected as are each R′, R″, R′″ and R″″ groups when more than one of these groups is present.

When R′ and R″ or R″ and R′″ are attached to the same nitrogen atom, they can be combined with the nitrogen atom to form a 4-, 5-, 6-, or 7-membered ring. For example, —NR′R″ is meant to include, but not be limited to, 1-pyrrolidinyl, 4 piperazinyl, and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, alkyl, heteroalkyl, alkenyl, alkynyl, cycloalkyl, heterocycloalkyl, aryl, arylalkyl, heteroaryl, heteroarylalkyl, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When an inhibitor of the invention includes more than one R group, for example, each of the R groups is independently selected as are each R′, R″, R′″ and R″″ groups when more than one of these groups is present.

As used herein, 0-2 in the context of —S(O)₍₀₋₂₎— are integers of 0, 1, and 2.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

The term “intermittent regimen” or “intermittent administration” refers to administration of a pharmaceutically active ingredient, (including but not limited to a PI3Kα inhibitor and/or an estrogen receptor antagonist) to a subject for at least one day, followed by an intermission or rest period of at least one day. For example, an intermittent regimen involves administering a pharmaceutically active ingredient for three consecutive days followed by a rest of 4 days in a 7-day treating period. In another example, an intermittent regimen involves administering a pharmaceutically active ingredient on three alternating days and not on days in between within a given treating period. In yet another example, an intermittent regimen involves administering a pharmaceutically active ingredient consecutively for at least 2, 3, 4, 5, 6, or more days, followed by an intermission, of at least 1, 2, 3, 4, 5, 6 or more days over a given treating period. In still another example, the pharmaceutically active ingredient can be administered on three alternating days and not administered on the days in between within the 7-day period.

The term “simultaneous” or “simultaneously” as applied to administering more than one pharmaceutically active ingredient (e.g., a PI3Kα inhibitor and an estrogen receptor antagonist) disclosed herein) refers to administering the more than one ingredient at the same time, or at two different time points that are separated by no more than 2 hours. The term “sequentially” as applied to administering more than one pharmaceutically active ingredient (e.g., a PI3Kα inhibitor and an estrogen receptor antagonist disclosed herein) refers to administering the more than one ingredient at two different time points that are separated by more than 2 hours, e.g., about 5 hours, 8 hours, 1 day, 2 days, 3 days, 4 days, 5 days, 6 days, 7 days or even longer.

As used herein, the term “intermission” refers to a period that is that subsequent to the administration of a particular pharmaceutically active ingredient, (including but not limited to a PI3Kα inhibitor and/or an estrogen receptor antagonist) of an intermittent regimen. Intermission refers to a rest period wherein a particular pharmaceutically active ingredient, (including but not limited to a PI3Kα inhibitor and/or an estrogen receptor antagonist) is not administered for at least one day.

The term “ALT” refers to the enzyme alanine aminotransferase.

The term “AST” refers to the enzyme aspartate aminotransferase.

The term “maximum tolerated dose” (MTD) refers to the maximum amount of a pharmaceutically active ingredient when administered as a simple agent, such as but not limited to a PI3Kα inhibitor, that can be administered to a subject without causing one or more non-tolerable adverse effects (e.g., adverse effects that are grade 3 or higher). Examples of adverse effects include but are not limited to increased levels of ALT and/or AST, e.g., beyond 5.0 fold of the upper limit of normal (ULN), nausea, diarrhoea, hyperglcaemia, vomiting, fatigue, decreased appetite, dry mouth, asthenia, dyspepsia, anaemia, thrombocytopenia, rash, leukopenia, stomatitis, decreased levels of insulin C-peptide, increased transaminases, decrease in weight, hypokalaemia, pruritus, abdominal pain, chills, and dysgeusia.

The term “area under the plasma concentration time curve” or “area under the curve” (AUC) refers to the integral, or area under the curve, in a plot of plasma concentration of drug against time. In some examples, the AUC is measured after administration of a pharmaceutically active ingredient, such as but not limited to a PI3Kα inhibitor, to assess bioavailability of the ingredient.

The terms “a week” or “7-day cycle”, as used herein, are used interchangeably. These terms refer to a continuous period of time covering the duration of seven consecutive days. For example, a week can start on Monday and end on the following Sunday, or a 7-day cycle can start on Wednesday and end on the next Tuesday.

Unless otherwise stated, structures depicted herein are also meant to include compounds which differ only in the presence of one or more isotopically enriched atoms. For example, compounds having the present structures wherein hydrogen is replaced by deuterium or tritium, or wherein carbon atom is replaced by ¹³C- or ¹⁴C-enriched carbon, are within the scope of this invention.

The present invention may include compounds that contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (⁴C). All isotopic variations of the compounds of the present invention, whether radioactive or not, are encompassed within the scope of the present invention. For example, the present invention may include pharmaceutically acceptable salts and radioactive isomers of compound A.

When ranges are used herein for physical properties, such as dose ranges, pharmacokinetic properties, such as Cₘₐₓ or AUC, and chemical properties, such as chemical formula, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

The PI3Kα inhibitors (e.g., compound A and its pharmaceutically acceptable salts and prodrugs) described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The present invention includes all manner of rotamers and conformationally restricted states of the PI3Kα inhibitor.

The PI3Kα inhibitor of the present invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

The PI3Kα inhibitor described herein can be optionally contacted with a pharmaceutically acceptable acid to form the corresponding acid addition salts. Pharmaceutically acceptable forms of the compounds recited herein include pharmaceutically acceptable salts, chelates, non-covalent complexes, prodrugs, and mixtures thereof. In certain embodiments, the PI3Kα inhibitor described herein are in the form of pharmaceutically acceptable salts. Those skilled in the art will recognize various synthetic methodologies that may be used to prepare non-toxic pharmaceutically acceptable addition salts.

### Treatment Regimens

In some embodiments, an intermittent treatment regimen of the invention achieves similar or better pathway inhibition than administering an equivalent dose of the PI3Kα inhibitor once daily. As used herein, the term “equivalent dose” refers to a single or multiple doses administered to a subject over a period of time, including a day, several days, a week, a month or longer. In some embodiments, equivalence is evaluated during the length of a treatment cycle, e.g. a week. The term equivalent dose is not limited to identical amounts of a compound administered of a specified period of time, but also refers to dose amounts which result in a similar level of tolerability. By way of example, when comparing a regimen of the invention in which a PI3Kα inhibitor is administered intermittently at a weekly cumulative dose of 1200 mg, with a regimen in which the PI3Kα inhibitor is administered daily, it may only possible to achieve a weekly cumulative dose of less than 1200 mg (e.g. about 1050 mg) using daily administration due to dose-limiting toxicity and/or limited tolerability. In such a case, administration of the weekly cumulative 1200 mg dose in the intermittent regimen is “equivalent” to the about 1050 mg weekly cumulative dose administered daily. In general, the difference between a weekly cumulative dose administered intermittently and daily can range from about 0% to about 20%.

Pathway inhibition may be measured, for example, as a percentage decrease in phosphorylation of a protein chosen from p4EBP1, pS6, and pRAS40. In some embodiments, pathway inhibition is measured as a 40%, 45%, 50%, 55%, 60%, 65%, 70%, 75%, 80%, 85%, 90%, 95% or greater decrease in phosphorylation of p4EBP1. For example, phosphorylation of p4EBP1 is reduced by at least 60%. In other embodiments, pathway inhibition is measured as a 40%, 45%, 50%, 55%, 60%, 65%, 70%, 75%, 80%, 85%, 90%, 95% or greater decrease in phosphorylation of pS6. For example, phosphorylation of pS6 is reduced by at least 60%. In yet other embodiments, pathway inhibition is measured as a 40%, 45%, 50%, 55%, 60%, 65%, 70%, 75%, 80%, 85%, 90%, 95% or greater decrease in phosphorylation of pRAS40. For example, phosphorylation of pRAS40 is reduced by at least 60%. In yet other embodiments, pathway inhibition is measured as a 40%, 45%, 50%, 55%, 60%, 65%, 70%, 75%, 80%, 85%, 90%, 95% or greater decrease in phosphorylation of p4EBP1, pS6, and pRAS40. For example, phosphorylation of p4EBP1, pS6, and pRAS40 is reduced by at least 60%. In some embodiments, pathway inhibition is measured in peripheral blood cells. In other embodiments, pathway inhibition is measured in a biopsy, for example a skin biopsy.

In some embodiments, an intermittent treatment regimen of the invention achieves similar or better level of tolerability as compared to administering an equivalent dose of the PI3Kα inhibitor once daily. The level of tolerability may be measured, for example, as the occurrence or lack of occurrence of a grade 3 or higher adverse event. In some embodiments, the adverse event is aspartate aminotransferase or alanine aminotransferase increase, rash, hyperglycaemia, lymphopenia, diarrhoea, gamma-glutamyltransferase increase, hypokalaemia, hyponatraemia, pruritus, thrombocytopenia, upper abdominal pain, anaemia, asthenia, catheter related infection, cellulitis, disease progression, enterocutaneous fistula, gastroenteritis, acute pancreatitis, pleural effusion, macular rash, somnolence, or urinary tract infection.

In some embodiments, an intermittent regimen of the invention allows for a weekly maximum tolerated dose (MTD) at least comparable to that obtained by daily administration when the total weekly dosage according to the intermittent regimen is comparable or less than that of the daily administration. In some embodiments, an intermittent regimen of the invention allows for a weekly maximum tolerated dose (MTD) greater than that obtained by daily administration when the total weekly dosage according to the intermittent regimen is comparable or less than that of the daily administration. In some embodiments, the total weekly dosage according to the daily administration regimen is higher than 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg or 3700 mg of a PI3Kα inhibitor disclosed herein.

In some embodiments, an intermittent regimen of the invention allows for a weekly maximum tolerated dose higher than about 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg or 3700 mg of the PI3Kα inhibitor.

In some embodiments, an intermittent regimen of the invention allows for a weekly maximum tolerated dosage from about 200 mg to about 3500 mg, about 300 mg to about 3600 mg, about 600 mg to about 3000 mg, about 900 mg to about 3000 mg, about 1200 mg to about 3000 mg, about 1200 mg to about 2700 mg, about 1800 mg to about 2700 mg of the PI3Kα inhibitor.

In some embodiments, an intermittent regimen of the invention allows for a weekly maximum tolerated dosage higher than about 600 mg of the PI3Kα inhibitor. In some embodiments, an intermittent regimen of the invention allows for a weekly maximum tolerated dosage higher than about 700 mg of the PI3Kα inhibitor. In other embodiments, an intermittent regimen of the invention allows for a weekly maximum tolerated dosage higher than about 1050 mg of the PI3Kα inhibitor. In yet other embodiments, an intermittent regimen of the invention allows for a weekly maximum tolerated dosage higher than about 1400 mg or about 1800 mg, or about 2100 mg, or about 2700 mg, or about 3000 mg of the PI3Kα inhibitor.

In some embodiments, the maximum tolerated dosage in a subject is determined by the observation of one or more adverse events. In some embodiments, the one or more adverse events is selected from the group consisting of increased levels of ALT and/or AST, nausea, diarrhoea, hyperglcaemia, vomiting, fatigue, decreased appetite, dry mouth, asthenia, dyspepsia, anaemia, thrombocytopenia, rash, leukopenia, stomatitis, decreased levels of insulin C-peptide, increased transaminases, decrease in weight, hypokalaemia, pruritus, abdominal pain, chills, and dysgeusia. In some embodiments, the adverse event is an increased level of ALT and/or AST.

In some embodiments, the maximum tolerated dosage in a subject is determined by an increase in ALT levels by more than about 1.25 times, about 2.5 times, about 5.0 times, or about 10 times the upper limit of normal (ULN). The ULN of ALT levels can be readily determined by one of ordinary skill in the art. For example, the ULN of ALT levels can be about 5 U/L, about 10 U/L, about 15 U/L 20 U/L, about 25 U/L, about 30 U/L, about 35 U/L about 40 U/L, about 45 U/L, about 50 U/L, about 55 U/L about 60 U/L, about 65 U/L, about 70 U/L, about 75 U/L about 80 U/L, about 85 U/L, about 90 U/L, about 95 U/L, or about 100 U/L.

In some embodiments, the maximum tolerated dosage in a subject is determined by an increase in AST levels by more than about 1.25 times, about 2.5 times, about 5.0 times, or about 10 times the upper limit of normal (ULN). The ULN of AST levels can be readily determined by one of ordinary skill in the art. For example, the ULN of AST levels can be about 5 U/L, about 10 U/L, about 15 U/L 20 U/L, about 25 U/L, about 30 U/L, about 35 U/L about 40 U/L, about 45 U/L, about 50 U/L, about 55 U/L about 60 U/L, about 65 U/L, about 70 U/L, about 75 U/L about 80 U/L, about 85 U/L, about 90 U/L, about 95 U/L, or about 100 U/L.

In some embodiments, the maximum tolerated dosage in a subject is determined by one or more adverse events that are grade 1 or higher. In some embodiments, the grade 1 or higher adverse event is an increase in ALT level by more than about 1.25 times the ULN. In some embodiments, the grade 1 or higher adverse event is an increase in AST level by more than about 1.25 times the ULN.

In some embodiments, the maximum tolerated dosage in a subject is determined by one or more adverse events that are grade 2 or higher. In some embodiments, the grade 2 or higher adverse event is an increase in ALT level by more than about 2.5 times the ULN. In some embodiments, the grade 2 or higher adverse event is an increase in AST level by more than about 2.5 times the ULN.

In some embodiments, the maximum tolerated dosage in a subject is determined by one or more adverse events that are grade 3 or higher. In some embodiments, a grade 3 or higher adverse event is an increase in ALT level by more than about 5.0 times the ULN. In some embodiments, a grade 3 or higher adverse event is an increase in AST level by more than about 5.0 times the ULN.

In some embodiments, the maximum tolerated dosage in a subject is determined by one or more adverse events that are grade 4 or higher. In some embodiments, a grade 4 or higher adverse event is an increase in ALT level by more than about 10.0 times the ULN. In some embodiments a grade 4 or higher adverse event is an increase in AST level by more than about 10.0 times the ULN.

The invention also provides a treatment regimen which is effective to achieve a Cₘₐₓ which is about ±20%, or about ±15% or about ±10% or about ±5% of the Cₘₐₓ achieved by administering an equivalent dose of the PI3Kα inhibitor once daily. For example, the Cₘₐₓ achieved is greater than about 0.2 μM, 0.5 μM, 1.0 μM, 2.0 μM, 4.0 μM, 6.0 μM, 8.0 μM, 10 μM, 15 μM, 20 μM, 25 μM, or 30 μM. In some instances, the Cₘₐₓ achieved is greater than about 1.0 μM, 2.0 μM, 4.0 μM, 6.0 μM, 8.0 μM, 10 μM, 15 μM, or 20 μM. For example, the Cₘₐₓ is greater than 2.0 μM. Alternatively, the Cₘₐₓ is greater than 4.0 μM. In other instances, the Cₘₐₓ achieved is between 4.0 μM and 20 μM. In yet other instances, the Cₘₐₓ achieved is between 6.0 μM and 20 μM. In yet other instances, the Cₘₐₓ achieved is between 6.0 μM and 15 μM.

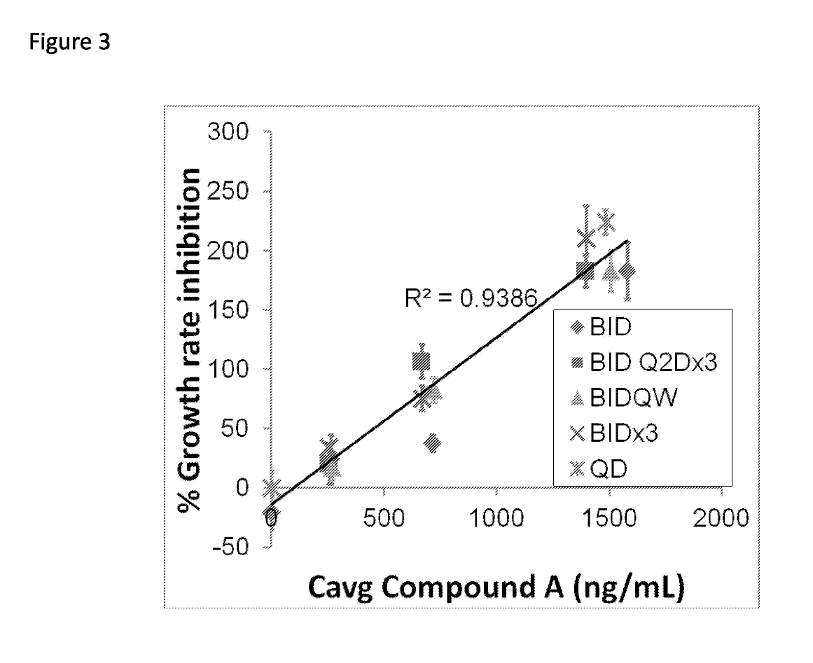

Preclinical data (see, e.g., FIG. 3) indicates that the efficacy of compound A is exposure (AUC)-driven. In some embodiments, an intermittent regimen of the invention allows for an area under the curve (AUC) at least comparable to that obtained by daily administration when the total weekly dosage according to the intermittent regimen is comparable or less than that of the daily administration. For example, an intermittent regimen of the invention allows for an area under the curve (AUC) at least comparable to that obtained by daily administration when the total weekly dosage according to the intermittent treatment regimen is comparable or less than that of the daily administration of about 600 mg, about 500 mg, about 400 mg, about 300 mg, about 250 mg, about 200 mg, about 150 mg, about 100 mg, about 50 mg, or about 10 mg.

In some embodiments, the intermittent regimen of the invention achieves an area under the curve (AUC) greater than about 1 μg*h/mL, about 2 μg*h/mL, about 3 μg*h/mL, about 4 μg*h/mL, about 5 μg*h/mL, about 6 μg*h/mL, about 7 μg*h/mL, about 8 μg*h/mL, about 9 μg*h/mL, about 10 μg*h/mL, about 20 μg*h/mL, about 30 μg*h/mL, about 40 μg*h/mL, about 50 μg*h/mL, about 100 μg*h/mL, about 150 μg*h/mL, about 200 μg*h/mL, about 250 μg*h/mL, about 300 μg*h/mL, about 400 μg*h/mL, about 500 μg*h/mL, about 600 μg*h/mL, about 700 μg*h/mL, about 800 μg*h/mL, about 900 μg*h/mL, about 1000 μg*h/mL, about 1500 μg*h/mL, or about 2000 μg*h/mL in the subject over a 7-day cycle. For example, the intermittent regimen of the invention achieves an AUC ranges from about 70 to about 500 μg*h/mL, or from about 100 to about 400 μg*h/mL, or from about 125 to about 300 μg*h/mL, or from about 125 to about 175 μg*h/mL in the subject over a 7-day cycle.

In some embodiments, the intermittent regimen of the invention achieves an area under the curve (AUC) greater than about 1 μg*h/mL, about 2 μg*h/mL, about 3 μg*h/mL, about 4 μg*h/mL, about 5 μg*h/mL, about 6 μg*h/mL, about 7 μg*h/mL, about 8 μg*h/mL, about 9 μg*h/mL, about 10 μg*h/mL, about 20 μg*h/mL, about 30 μg*h/mL, about 40 μg*h/mL, about 50 μg*h/mL, about 100 μg*h/mL, about 150 μg*h/mL, about 200 μg*h/mL, about 250 μg*h/mL, about 300 μg*h/mL, about 400 μg*h/mL, about 500 μg*h/mL, about 600 μg*h/mL, about 700 μg*h/mL, about 800 μg*h/mL, about 900 μg*h/mL, about 1000 μg*h/mL, about 1500 μg*h/mL, or about 2000 μg*h/mL in the subject over one dosing day. For example, the intermittent regimen of the invention achieves an AUC ranges from about 20 to about 180 μg*h/mL, or from about 30 to about 120 μg*h/mL, or from about 40 to about 80 μg*h/mL, or from about 45 to about 60 μg*h/mL in the subject over one dosing day.

In some embodiments, a given dosing schedule comprises one or more administrations of a PI3Kα inhibitor, wherein at least one administration of a PI3Kα inhibitor, such as described herein, may be repeated or cycled on a daily, weekly, biweekly, monthly, bimonthly, annually, semi-annually, or any other period. A repeated dosing schedule or cycle may be repeated for a fixed period of time determined at the start of the schedule; may be terminated, extended, or otherwise adjusted based on a measure of therapeutic effect, such as a level of reduction in the presence of detectable disease tissue (e.g. a reduction of at least 50%, 60%, 70%, 80%, 90%, 95%, 99%, or 100%); or may be terminated, extended, or otherwise adjusted for any other reason as determined by a medical professional.

In some embodiments, the PI3Kα inhibitor is administered on consecutive days in a 7-day cycle followed by an intermission. In some embodiments, the PI3Kα inhibitor is administered on alternate days in a 7-day cycle followed by an intermission.

In some embodiments, the intermittent regimen comprises at least one cycle in which the PI3Kα inhibitor is administered for at least 1 day, followed by an intermission in which the PI3Kα inhibitor is not administered for at least 1 day. For example, the PI3Kα inhibitor is administered for 1, 2, 3, 4, 5, 6 or 7 consecutive days followed by an intermission in which the PI3Kα inhibitor is not administered for at least 1 day, for example not administered for at least 1, 2, 3, 4, 5, 6, or 7 days. In some embodiments, the PI3Kα inhibitor is administered for 7, 8, 9, 10, 11, 12, 13, or 14 consecutive days, followed by an intermission where the PI3Kα inhibitor is not administered for at least 1, 2, 3, 4, 5, 6, or 7 days. In other embodiments, the PI3Kα inhibitor is administered for 2, 3, 4, 5, 6 or 7 consecutive days followed by an intermission in which the PI3Kα inhibitor is not administered for at least 3, 4, or 5 consecutive days. In yet other embodiments, the regimen comprises at least one 7-day cycle in which the PI3Kα inhibitor is administered for 3 consecutive days followed by an intermission of 4 consecutive days. For example, the PI3Kα inhibitor is administered once daily or twice daily every Monday, Tuesday, and Wednesday. In yet other embodiments, the regimen comprises at least one 7-day cycle in which the PI3Kα inhibitor is administered for 4 consecutive days followed by an intermission of 3 consecutive days. In yet other embodiments, the regimen comprises at least one 7-day cycle in which the PI3Kα inhibitor is administered for 5 consecutive days followed by an intermission of 2 consecutive days. In yet other embodiments, the regimen comprises at least one 7-day cycle in which the PI3Kα inhibitor is administered for 6 consecutive days followed by an intermission of 1 day.

In some embodiments, the intermittent regimen comprises at least one 7-day cycle in which the PI3Kα inhibitor is administered for at least 1 day. In some embodiments, the intermittent regimen comprises at least one 7-day cycle in which the PI3Kα inhibitor is administered for at least 2 days. In some embodiments, the intermittent regimen comprises at least one 7-day cycle in which the PI3Kα inhibitor is administered for at least 3 days. In some embodiments, the intermittent regimen comprises at least one 7-day cycle in which the PI3Kα inhibitor is administered for at least 4 days. In some embodiments, the intermittent regimen comprises at least one 7-day cycle in which the PI3Kα inhibitor is administered for at least 5 days. In some embodiments, the intermittent regimen comprises at least one 7-day cycle in which the PI3Kα inhibitor is administered for at least 6 days.

In some embodiments, the PI3Kα inhibitor is administered every other day (i.e., 7 dosing days in 2 weeks). In some embodiments, the PI3Kα inhibitor is administered on alternate days in a 7-day cycle. For example, the PI3Kα inhibitor is administered at least 2 times on alternate days within a 7-day cycle. In some embodiments, the PI3Kα inhibitor is administered at least 3 times on alternate days within a 7-day cycle. For example, the PI3Kα inhibitor is administered once daily or twice daily every Monday, Wednesday, and Friday. In some embodiments, the PI3Kα inhibitor is administered at least 4 times on alternate days within a 7-day cycle.

In some embodiments, the PI3Kα inhibitor is administered once a day (QD) in each of the days that the PI3Kα inhibitor is administered to the subject. In some embodiments, the PI3Kα inhibitor is administered twice a day (BID) in each of the days that the PI3Kα inhibitor is administered to the subject.

In some embodiments, a PI3Kα inhibitor and/or any additional therapeutic compound of the invention is administered in multiple doses. Dosing may be about once, twice, three times, four times, five times, six times, or more than six times per day or per week. Dosing may be about once a month, once every two weeks, once a week, or once every other day. In some embodiments, cycles of administering a PI3Kα inhibitor followed by periods of rest (intermission) are repeated for more than about 6, 10, 14, 28 days, two months, six months, or one year. In some cases, repetition of a dosing cycle comprising administration of a PI3Kα inhibitor followed by rest is continued as long as necessary. Administration of the treatment regimens of the invention may continue as long as necessary. In some embodiments, a PI3Kα inhibitor of the invention is administered for more than 1, 2, 3, 4, 5, 6, 7, 14, or 28 days. In some embodiments, a PI3Kα inhibitor of the invention is administered for less than 28, 14, 7, 6, 5, 4, 3, 2, or 1 day. In some embodiments, a PI3Kα inhibitor of the invention is administered chronically on an ongoing basis, e.g., for the treatment of chronic effects.

The amount of the PI3Kα inhibitor administered herein may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

A dosage form of the invention refers to the physical formulation of a drug for administration to the patient. When the dosage form is a solid, the dosage form can be a single capsule, tablet, or pill, or alternatively can be comprised of multiple capsules, tablets or pills. A dosage form may be administered to a subject once or multiple times per day. Methods of determining the most effective means and dosage of administration are well known to those of skill in the art and will vary with the composition used for therapy, the purpose of the therapy, the target cell or tissue being treated, and the subject being treated. Single or multiple administrations (e.g. about or more than about 1, 2, 3, 4, 5, 6, 7, 8, 9, 10, 11, 12, 13, 14, 15, 16, 17, 18, 19, 20, 25, 30, or more doses) can be carried out with the dose level and pattern being selected by the treating physician.

The PI3Kα inhibitor may be administered in any suitable amount. In some embodiments, the PI3Kα inhibitor is (6-(2-aminobenzo[d]oxazol-5-yl)imidazo[1,2-a]pyridin-3-yl)(morpholino)methanone and is administered to a subject within a range of about 600 mg to about 3000 mg, about 300 mg to about 3600 mg, about 900 mg to about 3000 mg, about 1200 mg to about 3000 mg, about 1200 mg to about 2700 mg, or about 1800 mg to about 2700 mg of the PI3Kα inhibitor per week. For example, this inhibitor is administered to a subject at a dosage of about 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600 mg per week. For another example, this inhibitor is administered to a subject at a dosage of about 1200, 1300, 1400, 1500, 1600, 1700, 1800, 1900, 2000, 2100, 2200, 2300, 2400, 2500, 2600, 2700, 2800, 2900, or 3000 mg per week. As another example, this inhibitor is administered to a subject at a dosage of about 1800, 1900, 2000, 2100, 2200, 2300, 2400, 2500, 2600, or 2700 mg per week.

In some embodiments, an inhibitor is administered to a subject in an amount greater than 50, 100, 150, 200, 250, 300, 400, 500, 600, 700, 800, 900, 1000, 1100, or 1200 mg per dosing day on average over the course of a treatment cycle. For example, the inhibitor is administered to a subject in an amount between about 100 and 1200 mg, between about 100 and 1000 mg, between about 100 and 900 mg, between about 150 and 900 mg, between about 150 and 600 mg, or between about 200 and 600 mg, or between about 200 and 400 mg on average over the course of a treatment cycle.

In some embodiments, an inhibitor is administered to a subject within a range of about 1 mg/kg-100 mg/kg per day, such as about, less than about, or more than about, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg per day. In some embodiments, an inhibitor is administered to a subject within a range of about 1 mg/kg-400 mg/kg per week, such as about, less than about, or more than about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, or 400 mg/kg per week. In some embodiments, an inhibitor is administered to a subject within a range of about 10 mg/kg-1500 mg/kg per month, such as about, less than about, or more than about 10 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg per month. The target dose may be administered in a single dose. Alternatively, the target dose may be administered in about or more than about 1, 2, 3, 4, 5, 6, 7, 8, 9, 10, 11, 12, 13, 14, 15, 16, 17, 18, 19, 20, 25, 30, or more doses. For example, a dose of about 20 mg/kg per week may be delivered weekly at a dose of about 20 mg/kg, or may be delivered at a dose of about 6.67 mg/kg administered on each of three days over the course of the week, which days may or may not be consecutive. The administration schedule may be repeated according to any regimen according to the invention, including any administration schedule described herein.

A dose of PI3Kα inhibitor may be about, at least about, or at most about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1175, 1100, 1125, 1150, 1175, 1200, 1225, 1250, 1275, or 1300 mg or mg/kg, or any range derivable therein. For example, such a dose can range from about 30-120 mg/kg (e.g., 40-80 mg/kg such as about 50 or about 60 mg/kg). It is contemplated that a dosage of mg/kg refers to the mg amount of inhibitor per kg of total body weight of the subject. It is contemplated that when multiple doses are given to a patient, the doses may vary in amount or they may be the same.

The amount of each inhibitor administered will be dependent on the mammal being treated, the severity of the disorder or condition, the rate of administration, the disposition of the compound and the discretion of the prescribing physician.

Methods and Treatment Regimens with an Estrogen Receptor Antagonist

In one aspect, the present invention provides a method of treating a neoplastic condition in a subject in need thereof comprising administering a therapeutically effective amount of a combination of a PI3-kinase α (PI3Kα) inhibitor and an estrogen receptor antagonist, including but not limited to fulvestrant. In some embodiments, the PI3Kα inhibitor is administered intermittently. In some embodiments, the neoplastic condition is a cancer selected from the group consisting of non-small cell lung cancer, small cell lung cancer, head and neck squamous cell carcinoma, pancreatic cancer, breast cancer, ovarian cancer, renal cell carcinoma, prostate cancer, neuroendocrine cancer, gastric cancer, bladder cancer, colon cancer and endometrial cancer. In some embodiments, the neoplastic condition is breast cancer.

In a separate but related aspect, the present invention provides for the treatment of a disorder mediated by PI3-kinase α (PI3Kα), wherein the regimen comprises administration of at least one estrogen receptor antagonist and at least one PI3Kα inhibitor to a human subject in need thereof.

In practicing any of the discussed methods, a PI3Kα inhibitor and an estrogen receptor antagonist can be administered sequentially, where the two agents are introduced into a subject at two different time points. The two time points can be separated by more than 2 hours, 1 or more days, 1 or more weeks, or according to any intermittent regimen schedule disclosed herein.

In certain embodiments, the PI3Kα inhibitor and the estrogen receptor antagonist are administered simultaneously. Simultaneous administration may take the format of co-administration of the two agents in same formulation, or in different formulations but at the same time.

As used herein, a therapeutically effective amount of a combination of a PI3Kα inhibitor and an estrogen receptor antagonist refers to a combination of a PI3Kα inhibitor and an estrogen receptor antagonist, wherein the combination is sufficient to effect the intended application including but not limited to disease treatment, as defined herein. Encompassed in this subject method is the use of therapeutically effective amount of a PI3Kα inhibitor and/or an estrogen receptor antagonist in combination to effect such treatment. Also contemplated in the subject methods is the use of a sub-therapeutic amount of a PI3Kα inhibitor and/or an estrogen receptor antagonist in the combination for treating an intended disease condition. The PI3Kα inhibitor and estrogen receptor antagonist individually, though present in sub-therapeutic amounts, synergistically yield an efficacious effect and/or reduced a side effect in an intended application.

The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In some embodiments, the PI3Kα inhibitor is administered according to an intermittent regimen. In some embodiments, the PI3Kα inhibitor is administered on consecutive days in a 7-day cycle followed by an intermission. In some embodiments, the PI3Kα inhibitor is administered on alternate days in a 7-day cycle followed by an intermission.

In some embodiments, the PI3Kα inhibitor is administered for at least 1 day, followed by an intermission in which the PI3Kα inhibitor is not administered for at least 1 day for at least one cycle. In some embodiments, the PI3Kα inhibitor is administered for 1, 2, 3, 4, 5, 6 or 7 consecutive days followed by an intermission in which the PI3Kα inhibitor is not administered for at least 1 day. In some embodiments, the PI3Kα inhibitor is administered for 1, 2, 3, 4, 5, 6 or 7 consecutive days followed by an intermission of at least 1, 2, 3, 4, 5, or 6 consecutive days. In some embodiments, the PI3Kα inhibitor is administered for 2, 3, 4, 5, 6 or 7 consecutive days followed by an intermission of at least 1, 2, 3, 4, 5, or 6 consecutive days. In some embodiments, the PI3Kα inhibitor is administered for 7, 8, 9, 10, 11, 12, 13, or 14 consecutive days, followed by an intermission where the PI3Kα inhibitor is not administered for at least 1, 2, 3, 4, 5, 6, or 7 days. In other embodiments, the PI3Kα inhibitor is administered for 2, 3, 4, 5, 6 or 7 consecutive days followed by an intermission in which the PI3Kα inhibitor is not administered for at least 3, 4, or 5 consecutive days.

In some embodiments, the PI3Kα inhibitor is administered on consecutive days in a 7-day cycle followed by an intermission. In yet other embodiments, the PI3Kα inhibitor is administered for 3 consecutive days followed by an intermission of 4 consecutive days for at least one 7-day cycle. In some embodiments, the PI3Kα inhibitor is administered for 3 consecutive days of a 7-day cycle followed by an intermission of at least one day. In some embodiments, the PI3Kα inhibitor is administered for 3 consecutive days followed by an intermission of 4 consecutive days per a 7-day cycle. In yet other embodiments, the PI3Kα inhibitor is administered for 4 consecutive days followed by an intermission of 3 consecutive days for at least one 7-day cycle. In yet other embodiments, the PI3Kα inhibitor is administered for 5 consecutive days followed by an intermission of 2 consecutive days for at least one 7-day cycle. In yet other embodiments, the PI3Kα inhibitor is administered for 6 consecutive days followed by an intermission of 1 day for at least one 7-day cycle.

In some embodiments, the PI3Kα inhibitor is administered for at least 1 day for at least one 7-day cycle. In some embodiments, the PI3Kα inhibitor is administered for at least 2 days for at least one 7-day cycle. In some embodiments, the PI3Kα inhibitor is administered for at least 3 days for at least one 7-day cycle. In some embodiments, the PI3Kα inhibitor is administered for at least 4 days for at least one 7-day cycle. In some embodiments, the PI3Kα inhibitor is administered for at least 5 days for at least one 7-day cycle. In some embodiments, the PI3Kα inhibitor is administered for at least 6 days for at least one 7-day cycle.

In some embodiments, the PI3Kα inhibitor is administered every other day (i.e., 7 dosing days in 2 weeks). In some embodiments, PI3Kα inhibitor is administered on three non-consecutive days of a 7-day cycle. In some embodiments, the PI3Kα inhibitor is administered on alternate days in a 7-day cycle. In some embodiments, the PI3Kα inhibitor is administered on alternate days in a 7-day cycle followed by an intermission. For example, the PI3Kα inhibitor is administered at least 2 times on alternate days within a 7-day cycle. In some embodiments, the PI3Kα inhibitor is administered at least 3 times on alternate days within a 7-day cycle. In some embodiments, the PI3Kα inhibitor is administered at least 4 times on alternate days within a 7-day cycle.

In some embodiments, the PI3Kα inhibitor is administered once a day (QD) in each of the days that the PI3Kα inhibitor is administered to the subject. In some embodiments, the PI3Kα inhibitor is administered twice a day (BID) in each of the days that the PI3Kα inhibitor is administered to the subject.

The PI3Kα inhibitor may be administered in any suitable amount. In some embodiments, the PI3Kα inhibitor is administered to a subject within a range of about 300 mg to about 3600 mg of the PI3Kα inhibitor in a 7-day cycle. In some embodiments, the PI3Kα inhibitor is administered to a subject within a range of about 900 mg to about 3600 mg of the PI3Kα inhibitor in a 7-day cycle. In some embodiments, the PI3Kα inhibitor is administered to a subject within a range of about 300 mg, about 900 mg to about 3600 mg of the PI3Kα inhibitor in a 7-day cycle. In some embodiments, the PI3Kα inhibitor is administered to a subject within a range of about 900 mg to about 3600 mg of the PI3Kα inhibitor in a 7-day cycle. For example, this inhibitor is administered to a subject at a dosage of about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3600 mg in a 7-day cycle. In some embodiments, the amount of PI3Kα inhibitor administered in a 7-day cycle is about 300 mg. In some embodiments, the amount of PI3Kα inhibitor administered in a 7-day cycle is about 900 mg. In some embodiments, the amount of PI3Kα inhibitor administered in a 7-day cycle is about 1800 mg. In some embodiments, the amount of PI3Kα inhibitor administered in a 7-day cycle is about 2700 mg. In some embodiments, the amount of PI3Kα inhibitor administered in a 7-day cycle is about 3600 mg.

In some embodiments, the dose used for a single administration of the PI3Kα inhibitor is about 100 to about 1200 mg. In some embodiments, the dose used for a single administration of the PI3Kα inhibitor is about 300 to about 1200 mg. In some embodiments, the dose used for a single administration of the PI3Kα inhibitor is about 100 mg, about 300 mg, about 600 mg, or about 900 mg. In some embodiments, the dose used for a single administration of the PI3Kα inhibitor is about 300 mg, about 600 mg, or about 900 mg. In some embodiments, the dose used for a single administration of the PI3Kα inhibitor is about 100 mg, about 300 mg, about 600 mg, about 900 mg, or about 1200 mg. In some embodiments, the dose used for a single administration of the PI3Kα inhibitor is about 300 mg, about 600 mg, about 900 mg, or about 1200 mg. In some embodiments, the dose used for a single administration of the PI3Kα inhibitor is about 100 mg. In some embodiments, the dose used for a single administration of the PI3Kα inhibitor is about 300 mg. In some embodiments, the dose used for a single administration of the PI3Kα inhibitor is about 600 mg. In some embodiments, the dose used for a single administration of the PI3Kα inhibitor is about 900 mg. In some embodiments, the dose used for a single administration of the PI3Kα inhibitor is about 1200 mg.

### PI3Kα Inhibitors

In one aspect, the PI3Kα inhibitor is a compound of the following formula:

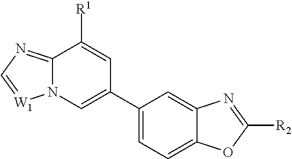

or a pharmaceutically acceptable salt thereof, wherein
W¹ is CR³;
R¹ is hydrogen;
R² is hydrogen, alkyl, heteroalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, sulfonamido, halo, cyano, hydroxy, nitro, phosphate, urea, carbonate, or NR′R″ wherein R′ and R″ are taken together with nitrogen to form a cyclic moiety; and
R³ is amido of formula —C(O)N(R)₂ or —NHC(O)R, wherein R is selected from the group consisting of hydrogen, alkyl, cycloalkyl, aryl, heteroaryl and heteroalicyclic; or wherein the (R)₂ groups taken together with the nitrogen to which it is attached form a 4-, 5-, 6-, or 7-membered ring.

In some embodiments, R³ is amido of formula —C(O)N(R)₂ wherein the (R)₂ groups taken together with the nitrogen to which they are attached form a 4-, 5-, 6-, or 7-membered ring. In a further embodiment, R³ is amido of formula —C(O)N(R)₂ wherein the (R)₂ groups taken together with the nitrogen to which they are attached form a 6-membered ring. In a further embodiment, R³ is amido of formula —C(O)N(R)₂ wherein the (R)₂ groups taken together with the nitrogen to which they are attached form a morpholinyl ring.

In some embodiments, R² is amino. In a further embodiment, R² is NH₂.

In some embodiments, R³ is amido of formula —C(O)N(R)₂ wherein the (R)₂ groups taken together with the nitrogen to which they are attached form a 4-, 5-, 6-, or 7-membered ring and R² is amino. In a further embodiment, R³ is amido of formula —C(O)N(R)₂ wherein the (R)₂ groups taken together with the nitrogen to which they are attached form a 6-membered ring and R² is amino. In a further embodiment, R³ is amido of formula —C(O)N(R)₂ wherein the (R)₂ groups taken together with the nitrogen to which they are attached form a morpholinyl ring and R² is NH₂.

In some embodiments, the PI3Kα inhibitor is a compound with the following structure:

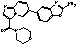 [SMILES: Nc1nc2cc(-c3ccc4ncc(C(=O)N5CCOCC5)n4c3)ccc2o1]

or a pharmaceutically acceptable salt thereof.

In some embodiments, the PI3Kα inhibitor is (6-(2-aminobenzo[d]oxazol-5-yl)imidazo[1,2-a]pyridin-3-yl)(morpholino)methanone) or compound A.

### Estrogen Receptor Antagonists

Estrogen receptor antagonists are agents that compete with estrogen for binding to the estrogen receptors. Estrogen promotes the cell proliferation in certain target tissues and has been linked to certain types of cancers, such as uterine and breast cancer. Accordingly, antiestrogens or estrogen receptor antagonists are used in the treatment of hormone receptor positive breast and uterine cancer. Estrogen receptor antagonists include selective estrogen receptor modulators (SERMs), which are agents that may function as estrogen-receptor agonists, antagonists or mixed agonists-antagonists and such activity is dependent on the target tissue. Examples of selective estrogen receptor modulators include and are not limited to raloxifene, tamoxifen, toremifene, droloxifene, idoxifene, arzoxifene, and EM-800. Estrogen receptor antagonists also include selective estrogen receptor downregulators (SERDs), which are competitive antagonists for every tissue targeted. Examples of selective estrogen downregulators (SERDs) include fulvestrant.

Fulvestrant, (7α,17β)-7-{9-[(4,4,5,5,5-pentafluoropentyl)sulfinyl]nonyl}estra-1,3,5(10)-triene-3,17-diol, down-regulates the estrogen receptor and is an estrogen receptor antagonist with no agonist effects. Fulvestrant is marketed by AstraZeneca under the name Faslodex® and is currently approved for the treatment of hormone receptor-positive metastatic breast cancer in postmenopausal women.

In some embodiments, the estrogen receptor antagonist is a selective estrogen receptor modulator. In some embodiments, the estrogen receptor antagonist is a selective estrogen receptor downregulator. In some embodiments, the estrogen receptor antagonist is raloxifene, tamoxifen, toremifene, droloxifene, idoxifene, arzoxifene, or fulvestrant. In some embodiments, the estrogen receptor antagonist is fulvestrant.

### Target Indications

For the target indications disclosed herein, the methods or treatment regimens involve administering a PI3Kα inhibitor for the treatment of the diseases as described. In some embodiments, these methods or treatment regimens involve administering a combination of a PI3Kα inhibitor and an estrogen receptor antagonist for any of the diseases or disorders disclosed herein.

The subject methods are useful for treating any disease conditions, for example diseases for which current treatment regimens result in adverse events, limited tolerability, or patient non-compliance. In some embodiments, the disease condition is a proliferative disorder, such as described herein, including but not limited to cancer. In other embodiments, the disorder is diabetes. In still other embodiments, the disorder is an autoimmune disorder.

In some embodiments, the disease condition is associated with PI3-kinase and/or mTOR. A vast diversity of disease conditions associated with PI3-kinase and/or mTOR have been reported. PI3-kinase α, one of the four isoforms of type I PI3-kinases has been implicated, for example, in a variety of human proliferative disorders, such as cancers. Angiogenesis has been shown to selectively require PI3Kα in the control of endothelial cell migration. (Graupera et al, Nature 2008; 453; 662-6). Mutations in the gene coding for PI3K a or mutations which lead to upregulation of PI3K α are believed to occur in many human cancers such as lung, stomach, endometrial, ovarian, bladder, breast, colon, brain and skin cancers. Often, mutations in the gene coding for PI3K α are point mutations clustered within several hotspots in helical and kinase domains, such as E542K, E545K, and H1047R. Many of these mutations have been shown to be oncogenic gain-of-function mutations. Because of the high rate of PI3K α mutations, targeting of this pathway provides valuable therapeutic opportunities. While other PI3K isoforms such as PI3K δ or PI3K γ are expressed primarily in hematopoietic cells, PI3K α, along with PI3K β, is expressed constitutively.

Disease conditions associated with PI3-kinase and/or mTOR can also be characterized by abnormally high level of activity and/or expression of downstream messengers of mTOR and PI3-kinase. For example, proteins or messengers such as PIP2, PIP3, PDK, Akt, PTEN, PRAS40, GSK-3β, p21, p27 may be present in abnormal amounts which can be identified by any assays known in the art.

Deregulation of the PI3K/Akt/mTOR pathway is emerging as a common theme in diverse human diseases and as a consequence drugs that target PI3Kα have therapeutic value. The diseases associated with deregulation of PI3Kα include, but are not limited to, tuberous sclerosis complex (TSC) and lymphangioleiomyomatosis (LAM), both of which are caused by mutations in TSC1 or TSC2 tumor suppressors. Patients with TSC develop benign tumors that when present in brain, however, can cause seizures, mental retardation and death. LAM is a serious lung disease Inhibition of PI3Kα may help patients with Peutz-Jeghers cancer-prone syndrome caused by the LKB 1 mutation. PI3Kα may also have role in the genesis of sporadic cancers. Inactivation of several tumor suppressors, in particular PTEN, p53, VHL and NF1, has been linked to mTORC1 activation. The PI3K/Akt/mTOR pathway is activated in many cancers. Activated Akt regulates cell survival, cell proliferation and metabolism by phosphorylating proteins such as BAD, FOXO, NF-KB, p21Cip1, p27Kip1, GSK3β and others. Akt might also promote cell growth by phosphorylating TSC2. Akt activation may promote cellular transformation and resistance to apoptosis by collectively promoting growth, proliferation and survival, while inhibiting apoptotic pathways.

Where desired, the subject to be treated is tested prior to treatment using a diagnostic assay to determine the sensitivity of tumor cells to a PI3Kα inhibitor. Any method known in the art that can determine the sensitivity of the tumor cells of a subject to a PI3Kα inhibitor can be employed. In these methods one or more additional anti-cancer agents or treatments can be co-administered according to a treatment regimen of the invention using the PI3Kα inhibitor, as judged to be appropriate by the administering physician given the prediction of the likely responsiveness of the subject to the combination of the PI3Kα inhibitor, in combination with any additional circumstances pertaining to the individual subject. In some embodiments, the anti-cancer agent is fulvestrant. In some embodiments, the PI3Kα inhibitor is used in combination with an estrogen receptor antagonist simultaneous or sequential manner.

The data presented in the Examples herein below demonstrate that the anti-tumor effects of an intermittent regimen of the invention involving an agent which is a PI3Kα inhibitor (where the PI3Kα inhibitor is administered according to a treatment regimen or method disclosed herein) are superior to the anti-tumor effects of the agent administered daily. As such, the subject methods are particularly useful for treating a proliferative disorder, such as a neoplastic condition. Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the disorder is a cancer selected from the group consisting of non-small cell lung cancer, small cell lung cancer, head and neck squamous cell carcinoma, pancreatic cancer, breast cancer, ovarian cancer, renal cell carcinoma, prostate cancer, neuroendocrine cancer, gastric cancer, bladder cancer, colon cancer and endometrial cancer. In some embodiments, the disease is breast cancer. In some embodiments, the disease is hormone receptor positive breast cancer.

The determination of the hormone receptor status of breast cancer is important in planning the course of treatment. Specifically, the assessment of estrogen receptor (ER) and/or progesterone receptor (PR) expression of the tumor is useful in predicting if the cancer will be responsive to hormone therapy. A cancer that is estrogen receptor (ER) positive and/or progesterone receptor (PR) positive suggests that the hormone therapy, such as those utilizing antiestrogens or estrogen receptor antagonists, may inhibit tumor growth. Furthermore, the assesment of HER2/neu (human epidermal growth factor receptor 2) expression in breast cancer, is also useful in planning the course of treatment as the overexpression of HER2 has been linked with cancer cell proliferation. A cancer that is determined to Her/neu positive indicates that the cancer will be responsive to therapies that specifically target HER2.

In some embodiments, the breast cancer is estrogen receptor (ER) positive breast cancer. In some embodiments, the breast cancer is positive for estrogen receptor expression as determined by a hormone receptor assay. In some embodiments, the breast cancer is estrogen receptor (ER) positive, progesterone receptor (PR) positive, and human epidermal growth factor receptor 2 (HER2) positive breast cancer. In some embodiments, the breast cancer is positive for the expression of estrogen receptor (ER), progesterone receptor (PR) and human epidermal growth factor receptor 2 (HER2) as determined by a hormone receptor assay.

In some embodiments, a treatment regimen involves administering a PI3Kα inhibitor for the treatment of a cancer which is lung cancer, breast cancer, endometrial cancer, ovarian cancer, bladder cancer, prostate cancer, neuroendocrine cancer, renal cancer, lymphoma, myeloma or leukemia. In some embodiments, a treatment regimen involves administering simultaneously or sequentially a combination of a PI3Kα inhibitor and an estrogen receptor antagonist for the treatment of a cancer.

In some embodiments, a treatment regimen involves administering a PI3Kα inhibitor for the treatment of solid tumors. In some embodiments, a treatment regimen involves administering a combination of a PI3Kα inhibitor and an estrogen receptor antagonist for the treatment of solid tumors. Solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Additional exemplary solid tumors include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In some embodiments, a treatment regimen of the invention involves administering a PI3Kα inhibitor for the treatment of multiple myeloma and/or Waldenstrom's macroglobulinemia.

In some embodiments, a treatment regimen involves administering a PI3Kα inhibitor for the treatment of renal cell carcinoma (also known as RCC or hypernephroma). Renal cell carcinoma is a kidney cancer that originates in the lining of the proximal convoluted tubule. Any known type of renal cell carcinoma may be treated using the treatment regimens of the invention, including clear renal cell carcinoma, papillary renal cell carcinoma, chromophobe renal cell carcinoma and collecting duct carcinoma. Any stage of the disease may be treated using the methods of the invention, including early stage as well as later stages (e.g. metastatic renal cell carcinoma).

In other embodiments, the treatment regimen involves administering a PI3Kα inhibitor for treatment of heart conditions including atherosclerosis, heart hypertrophy, cardiac myocyte dysfunction, elevated blood pressure and vasoconstriction. The invention also relates to a method of treating diseases related to vasculogenesis or angiogenesis in a mammal that comprises subjecting said mammal to a therapeutically effective regimen using a PI3Kα inhibitor of the present invention, or any pharmaceutically acceptable salt, ester, prodrug, solvate, hydrate or derivative thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, sarcoma and ovarian, breast, lung, pancreatic, prostate, colon and epidermoid cancer.

In some embodiments, the invention provides a treatment regimen involving administering a PI3Kα inhibitor for treating a disease condition associated with PI3-kinase α and/or mTOR, including, but not limited to, conditions related to an undesirable, over-active, harmful or deleterious immune response in a mammal, collectively termed “autoimmune disease.” Autoimmune disorders include, but are not limited to, Crohn's disease, ulcerative colitis, psoriasis, psoriatic arthritis, juvenile arthritis and ankylosing spondilitis, Other non-limiting examples of autoimmune disorders include autoimmune diabetes, multiple sclerosis, systemic lupus erythematosus (SLE), rheumatoid spondylitis, gouty arthritis, allergy, autoimmune uveitis, nephrotic syndrome, multisystem autoimmune diseases, autoimmune hearing loss, adult respiratory distress syndrome, shock lung, chronic pulmonary inflammatory disease, pulmonary sarcoidosis, pulmonary fibrosis, silicosis, idiopathic interstitial lung disease, chronic obstructive pulmonary disease, asthma, restenosis, spondyloarthropathies, Reiter's syndrome, autoimmune hepatitis, inflammatory skin disorders, vasculitis oflarge vessels, medium vessels or small vessels, endometriosis, prostatitis and Sjogren's syndrome. Undesirable immune response can also be associated with or result in, e.g., asthma, emphysema, bronchitis, psoriasis, allergy, anaphylaxsis, autoimmune diseases, rhuematoid arthritis, graft versus host disease, transplantation rejection, lung injuries, and lupus erythematosus. The pharmaceutical compositions of the present invention can be used to treat other respiratory diseases including but not limited to diseases affecting the lobes of lung, pleural cavity, bronchial tubes, trachea, upper respiratory tract, or the nerves and muscle for breathing. The methods of the invention can be further used to treat multiorgan failure.

The invention also provides a treatment regimen involving administering a PI3Kα inhibitor for treating liver diseases (including diabetes), pancreatitis or kidney disease (including proliferative glomerulonephritis and diabetes-induced renal disease) or pain in a mammal.

The invention further provides a treatment regimen involving administering a PI3Kα inhibitor for treating sperm motility. The invention also provides a treatment regimen involving administering a an PI3Kα inhibitor for treating neurological or neurodegenerative diseases including, but not limited to, Alzheimer's disease, Huntington's disease, CNS trauma, and stroke.

The invention further provides a treatment regimen involving administering a PI3Kα inhibitor for the prevention of blastocyte implantation in a mammal.

The invention also relates to a treatment regimen involving administering a PI3Kα inhibitor for treating a disease related to vasculogenesis or angiogenesis in a mammal which can manifest as tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, hemangioma, glioma, melanoma, sarcoma and ovarian, breast, lung, pancreatic, prostate, colon and epidermoid cancer.

The invention further provides a treatment regimen involving administering a PI3Kα inhibitor for the treatment of disorders involving platelet aggregation or platelet adhesion, including but not limited to Bernard-Soulier syndrome, Glanzmann's thrombasthenia, Scott's syndrome, von Willebrand disease, Hermansky-Pudlak Syndrome, and Gray platelet syndrome.

In some embodiments, a treatment regimen is provided involving administering a PI3Kα inhibitor to treat disease which is skeletal muscle atrophy, skeletal muscle hypertrophy, leukocyte recruitment in cancer tissue, invasion metastasis, melanoma, sarcoma, acute and chronic bacterial and viral infections, sepsis, glomerulo sclerosis, glomerulo, nephritis, or progressive renal fibrosis.

Certain embodiments contemplate a human subject such as a subject that has been diagnosed as having or being at risk for developing or acquiring a proliferative disorder condition. Certain other embodiments contemplate a non-human subject, for example a non-human primate such as a macaque, chimpanzee, gorilla, vervet, orangutan, baboon or other non-human primate, including such non-human subjects that can be known to the art as preclinical models, including preclinical models for inflammatory disorders. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present invention, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

### Therapeutic Efficacy

In some embodiments, therapeutic efficacy is measured based on an effect of treating a proliferative disorder, such as cancer. In general, therapeutic efficacy of the methods and compositions of the invention, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, and/or a reduction in the size of at least one tumor such that a human is treated for the proliferative disorder. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. The primary efficacy parameter used to evaluate the treatment of cancer by the inventive method and compositions preferably is a reduction in the size of a tumor. Tumor size can be figured using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

In some embodiments, tumor size is reduced as a result of the inventive method preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. The NCI toxicity scale (published April 1999) and Common Toxicity Criteria Manual (updated August 1999) is available through the NCI, e.g., through the NCI internet website at www.ctep.info.nih.gov or in the Investigator's Handbook for participants in clinical trials of investigational agents sponsored by the Division of Cancer Treatment and Diagnosis, NCI. Desirably, the inventive method is associated with minimal adverse events, e.g. Grade 0, Grade 1, or Grade 2 adverse events, as graded by the CTEP/NCI.

As discussed herein, reduction of tumor size, although preferred, is not required in that the actual size of tumor may not shrink despite the eradication of tumor cells. Eradication of cancerous cells is sufficient to realize a therapeutic effect. Likewise, any reduction in tumor size is sufficient to realize a therapeutic effect.

Desirably, the growth of a tumor is stabilized (i.e., one or more tumors do not increase more than 1%, 5%, 10%, 15%, or 20% in size, and/or do not metastasize) as a result of the inventive method and compositions. Such stabilization may be evidenced by a longer period of stable disease as characterized by the RECIST guidelines. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. Preferably, the inventive method reduces the size of a tumor at least about 5% (e.g., at least about 10%, 15%, 20%, or 25%). More preferably, tumor size is reduced at least about 30% (e.g., at least about 35%, 40%, 45%, 50%, 55%, 60%, or 65%). Even more preferably, tumor size is reduced at least about 70% (e.g., at least about 75%, 80%, 85%, 90%, or 95%). Most preferably, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, no detectable amount of tumor is found in the subject for at least about 1, 2, 3, 4, 5, 6, 7, 8, 9, 10, or more years after the treatment.

When a tumor is subject to surgical resection following completion of the therapeutic period, the efficacy of the inventive method in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is 100%, that is, no tumor tissue is present or detectable.

A number of secondary parameters can be employed to determine the efficacy of the inventive method. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also efficiently distinguishes small tumors from surrounding tissue (see, for example, U.S. Pat. No. 4,932,412).

Desirably, in accordance with the inventive method, the treatment of cancer in a human patient is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period or (e) a longer period of stable disease, for example longer by 1, 2, 3, 4, or 5 months. While at least a 2% decrease in a tumor-associated antigen level is preferred, any decrease in the tumor-associated antigen level is evidence of treatment of a cancer in a patient by the inventive method. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with the invention can be evidenced in terms of pain intensity, analgesic consumption, and/or the Karnofsky Performance Scale score. The Karnofsky Performance Scale allows patients to be classified according to their functional impairment. The Karnofsky Performance Scale is scored from 0-100. In general, a lower Karnofsky score is predictive of a poor prognosis for survival. Thus, the treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

Detection, monitoring, and rating of various cancers in a human are further described in Cancer Facts and Figures 2001, American Cancer Society, New York, N.Y., and International Patent Application WO 01/24684. Accordingly, a clinician can use standard tests to determine the efficacy of the various embodiments of the inventive method in treating cancer. However, in addition to tumor size and spread, the clinician also may consider quality of life and survival of the subject in evaluating efficacy of treatment.

In some embodiments, administration of a PI3Kα inhibitor according to an intermittent regiment of the invention provides improved therapeutic efficacy over a treatment where the inhibitor is administered daily. Improved efficacy may be measured using any method known in the art, including but not limited to those described herein. In some embodiments, the improved therapeutic efficacy is an improvement of at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000%, 10000% or more, using an appropriate measure (e.g. tumor size reduction, duration of tumor size stability, duration of time free from metastatic events, duration of disease-free survival). Improved efficacy may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or more, using an appropriate measure (e.g. tumor size reduction, duration of tumor size stability, duration of time free from metastatic events, duration of disease-free survival).

### Pharmaceutical Compositions and Administration

In one aspect, the subject pharmaceutical composition comprises a PI3Kα inhibitor.

In another aspect, the pharmaceutical composition provides for a combination treatment utilizing a PI3Kα inhibitor and an estrogen receptor antagonist. The therapeutic agents (including compounds) that are provided for use in the combination therapies of the invention can be administered simultaneously or separately. This administration in combination includes, for example, simultaneous administration of two agents in the same dosage form, simultaneous administration in separate dosage forms, and separate administration. For example, multiple therapeutic agents can be formulated together in the same dosage form and administered simultaneously. Alternatively multiple therapeutic agents can be simultaneously administered, wherein both the agents are present in separate formulations. In another alternative, a compound of the present invention can be administered just followed by and any of the agents described above, or vice versa. In the separate administration protocol, a compound of the present invention and any of the agents described above may be administered a few minutes apart, or a few hours apart, or a few days apart. The term “combination treatments” also embraces the administration of the therapeutic agents as described herein in further combination with other biologically active compounds or ingredients and non-drug therapies (e.g., surgery or radiation treatment).

Administration of the compounds of the present invention can be effected by any method that enables delivery of the compounds to the site of action. An effective amount of an inhibitor of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Sequential or substantially simultaneous administration of each inhibitor or therapeutic agent can be effected by any appropriate route as noted above and including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

In some embodiments, administration of the compounds of the invention can be effected in one dose, continuously or intermittently throughout the course of treatment. Methods of determining the most effective means and dosage of administration are well known to those of skill in the art and will vary with the composition used for therapy, the purpose of the therapy, the target cell or tissue being treated, and the subject being treated. Single or multiple administrations can be carried out with the dose level and pattern being selected by the treating physician.

The amount of each compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a combination treatment of the invention is administered in a single dose comprising at least a PI3Kα inhibitor and an estrogen receptor antagonist. Typically, such administration will be by injection, e.g., intravenous injection, in order to introduce the agent quickly. However, other routes may be used as appropriate. A single dose of a combination treatment of the invention may also be used for treatment of an acute condition.

When a combination treatment of the invention is administered as a composition that comprises one or more compounds, and one compound has a shorter half-life than another compound, the unit dose forms may be adjusted accordingly.

The subject pharmaceutical compositions can be administered as a combination of a PI3Kα inhibitor and an estrogen receptor antagonist, or in further combination with one or more other agents, which are also typically administered in the form of pharmaceutical compositions. Where desired, the subject combinations and other agent(s) may be mixed into a preparation or both components may be formulated into separate preparations to use them in combination separately or at the same time.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

The subject pharmaceutical compositions are typically formulated to provide a therapeutically effective amount of a compound of the present invention as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (e.g., a compound) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the invention provides a solid pharmaceutical composition for oral administration containing: (i) a compound which is a PI3Kα inhibitor; (ii) a second compound which is an estrogen receptor antagonist; and (iii) a pharmaceutical excipient suitable for oral administration. In some embodiments, the composition further contains: (iv) a third agent or even a fourth agent. In some embodiments, each compound or agent is present in a therapeutically effective amount. In other embodiments, one or more compounds or agents is present in a sub-therapeutic amount, and the compounds or agents act synergistically to provide a therapeutically effective pharmaceutical composition.

In some embodiments, the invention provides for a pharmaceutical composition comprising a combination of a PI3Kα inhibitor and an estrogen receptor antagonist. The PI3-kinase α inhibitor and the estrogen receptor antagonist can be packaged as a single oral dosage form. In other embodiments, the PI3Kα inhibitor and the estrogen receptor antagonist can be packaged as separate dosage forms, such as a tablet.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, hydroxypropyl cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. In some embodiments, the pharmaceutical composition for injection comprises at least one PI3Kα inhibitor and at least one estrogen receptor antagonist. Also provided are pharmaceutical compositions comprising a PI3Kα inhibitor, and pharmaceutical compositions comprising an estrogen receptor antagonist, where the PI3Kα inhibitor is administered separately or together with the estrogen receptor antagonist. The PI3Kα inhibitor and the estrogren receptor antagonist may be formulated separately, and may further include a third therapeutic agent. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery

In some embodiments, the invention provides a pharmaceutical composition for transdermal delivery containing a compound of the present invention and a pharmaceutical excipient suitable for transdermal delivery. In some embodiments, the pharmaceutical composition for topical delivery comprises at least one PI3Kα inhibitor and at least one estrogen receptor antagonist.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. bydividing such larger doses into several small doses for administration throughout the day.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. No. 5,451,233; U.S. Pat. No. 5,040,548; U.S. Pat. No. 5,061,273; U.S. Pat. No. 5,496,346; U.S. Pat. No. 5,292,331; U.S. Pat. No. 5,674,278; U.S. Pat. No. 3,657,744; U.S. Pat. No. 4,739,762; U.S. Pat. No. 5,195,984; U.S. Pat. No. 5,292,331; U.S. Pat. No. 5,674,278; U.S. Pat. No. 5,879,382; U.S. Pat. No. 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include a compound or compounds of the present invention as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Combination Therapies

In one aspect, the present invention also provides methods for further combination therapies in which, in addition to an PI3Kα inhibitor, one or more agents known to modulate other pathways, or other components of the same pathway, or even overlapping sets of target enzymes is used or a pharmaceutically acceptable salt, ester, prodrug, solvate, hydrate or derivative thereof. In another aspect, the present invention also provides methods for further combination therapies in which, in addition to an PI3Kα inhibitor and an estrogen receptor antagonist, one or more agents known to modulate other pathways, or other components of the same pathway, or even overlapping sets of target enzymes is used or a pharmaceutically acceptable salt, ester, prodrug, solvate, hydrate or derivative thereof. In one aspect, such therapy includes but is not limited to the combination of the composition comprising a PI3Kα inhibitor, as described herein, with one or more additional therapeutic agents such as anticancer agents, chemotherapeutic agents, therapeutic antibodies, and radiation treatment, to provide, where desired, a synergistic or additive therapeutic effect. Pathways that may be targeted by administering another agent include, but are not limited to, MAP kinase, Akt, NFkB, WNT, RAS/RAF/MEK/ERK, JNK/SAPK, p38 MAPK, Src Family Kinases, JAK/STAT and/or PKC signaling pathways. Other agents may target one or more members of one or more signaling pathways. Representative members of the nuclear factor-kappaB (NFkB) pathway include but are not limited to RelA (p65), RelB, c-Rel, p50/p105 (NF-κB 1), p52/p 100 (NF-κB2), IkB, and IkB kinase. Non-limiting examples of receptor tyrosine kinases that are members of the phosphatidylinositol 3-kinase (PI3K)/AKT pathway that may be targeted by one or more agents include FLT3 LIGAND, EGFR, IGF-1R, HER2/neu, VEGFR, and PDGFR. Downstream members of the PI3K/AKT pathway that may be targeted by agents according to the methods of the invention include, but are not limited to, forkhead box O transcription factors, Bad, GSK-3β, I-κB, mTOR, MDM-2, and S6 ribosomal subunit.

Anticancer agents useful in the methods of the invention include but are not limited to paclitaxel, fulvestrant, exemestane, gemcitabine, erlotinib, gefitinib, afatinib, nintedanib, dacomitinib, bevacizumab, pemetrexed, motesanib, crizotinib, ipilimumab, ramucirumab, custirsen, and onartuzumab.

Other agents useful in the methods of the invention include any capable of modulating a target molecule, either directly or indirectly. Non-limiting examples of target molecules modulated by other agents include enzymes, enzyme substrates, products of transitions, antibodies, antigens, membrane proteins, nuclear proteins, cytosolic proteins, mitochondrial proteins, lysosomal proteins, scaffold proteins, lipid rafts, phosphoproteins, glycoproteins, membrane receptors, G-protein-coupled receptors, nuclear receptors, protein tyrosine kinases, protein serine/threonine kinases, phosphatases, proteases, hydrolases, lipases, phospholipases, ligases, reductases, oxidases, synthases, transcription factors, ion channels, RNA, DNA, RNAse, DNAse, phospholipids, sphingolipids, nuclear receptors, ion channel proteins, nucleotide-binding proteins, calcium-binding proteins, chaperones, DNA binding proteins, RNA binding proteins, scaffold proteins, tumor suppressors, cell cycle protei3ns, and histones.

Other agents may target one or more signaling molecules including but not limited to the following: HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tp1, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon β, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, Cdk4, Cdk 6, Cdk 2, Cdkl, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, \tilde{β}-catenin, FOXO STAT1, STAT 3, STAT 4, STAT 5, STAT 6, p53, WT-1, HMGA, pS6, 4EPB-1, eIF4E-binding protein, RNA polymerase, initiation factors, and elongation factors.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned herein, for example as potentiators of therapeutic activity of such drugs or as a means of reducing required dosaging or potential side effects of such drugs. An inhibitor of the invention may be mixed with the other drug substance in a fixed pharmaceutical composition or it may be administered separately, before, simultaneously with or after the other drug substance. Accordingly the invention includes a combination of an inhibitor of the invention as described with an anti-inflammatory, bronchodilatory, antihistamine or anti-tussive drug substance, said compound of the invention and said drug substance being in the same or different pharmaceutical composition. Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticoid receptor agonists such as those described in WO 00/00531, WO 02/10143, WO 03/082280, WO 03/082787, WO 03/104195, WO 04/005229; LTB4 antagonists such LY29311 1, CGS025019C, CP-195543, SC-53228, BIIL 284, ONO 4057, SB 209247 and those described in U.S. Pat. No. 5,451,700; LTD4 antagonists such as montelukast and zafirlukast; PDE4 inhibitors such cilomilast (Ariflo® GlaxoSmithKline), Roflumilast (Byk Gulden), V-1 1294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, as well as compounds (in free or salt or solvate form) of formula I of WO 04/16601, and also compounds of WO 04/033412. Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/041 18, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

Other useful combinations of compounds of the invention with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351 125, SCH-55700 and SCH-D, Takeda antagonists such as TAK-770, and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

The compounds of the invention may be formulated or administered in conjunction with other agents that act to relieve the symptoms of inflammatory conditions such as encephalomyelitis, asthma, and the other diseases described herein. These agents include non-steroidal anti-inflammatory drugs (NSAIDs), e.g., acetylsalicylic acid; ibuprofen; naproxen; indomethacin; nabumetone; tolmetin; etc. Corticosteroids are used to reduce inflammation and suppress activity of the immune system. The most commonly prescribed drug of this type is Prednisone. Chloroquine (Aralen) or hydroxychloroquine (Plaquenil) may also be very useful in some individuals with lupus. They are most often prescribed for skin and joint symptoms of lupus. Azathioprine (Imuran) and cyclophosphamide (Cytoxan) suppress inflammation and tend to suppress the immune system. Other agents, e.g., methotrexate and cyclosporin are used to control the symptoms of lupus. Anticoagulants are employed to prevent blood from clotting rapidly. They range from aspirin at very low dose which prevents platelets from sticking, to heparin/coumadin.

In one aspect, this invention also relates to methods and pharmaceutical compositions for inhibiting abnormal cell growth in a mammal which comprises an amount of a PI3Kα inhibitor of the present invention, or a pharmaceutically acceptable salt, ester, prodrug, solvate, hydrate or derivative thereof, in combination with an amount of an anti-cancer agent (e.g., a chemotherapeutic agent). Many chemotherapeutics are presently known in the art and can be used in combination with the compounds of the invention.

This invention further relates to a method for using the compounds or pharmaceutical composition in combination with other tumor treatment approaches, including surgery, ionizing radiation, photodynamic therapy, or implants, e.g., with corticosteroids, hormones, or used as radiosensitizers.

One such approach may be, for example, radiation therapy in inhibiting abnormal cell growth or treating the proliferative disorder in the mammal. Techniques for administering radiation therapy are known in the art, and these techniques can be used in the combination therapy described herein. The administration of the compound of the invention in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

Without being limited by any theory, the compounds of the present invention can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a PI3Kα inhibitor of the present invention, or a pharmaceutically acceptable salt, ester, prodrug, solvate, hydrate or derivative thereof, which combined amounts are effective in sensitizing abnormal cells to treatment with radiation. The amount of the compound, salt, or solvate in this method can be determined according to the means for ascertaining effective amounts of such compounds described herein.

Photodynamic therapy includes therapy which uses certain chemicals known as photosensitizing compounds to treat or prevent cancers. Examples of photodynamic therapy include treatment with compounds, such as e.g., VISUDYNE and porfimer sodium. Angiostatic steroids include compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids include compounds, such as e.g., fluocinolone and dexamethasone. Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The invention also relates to a method of and to a pharmaceutical composition of treating a cardiovascular disease in a mammal which comprises administering an amount of a PI3Kα inhibitor of the present invention, or a pharmaceutically acceptable salt, ester, prodrug, solvate, hydrate or derivative thereof, or an isotopically-labeled derivative thereof, and, separately or in combination with the PI3Kα inhibitor, administering an amount of one or more therapeutic agents useful for the treatment of cardiovascular diseases. The invention also relates to a method of and to a pharmaceutical composition which comprises administering a combination of a PI3Kα inhibitor and an estrogen receptor antagonist.

Exemplary agents for use in cardiovascular disease applications are anti-thrombotic agents, e.g., prostacyclin and salicylates, thrombolytic agents, e.g., streptokinase, urokinase, tissue plasminogen activator (TPA) and anisoylated plasminogen-streptokinase activator complex (APSAC), anti-platelets agents, e.g., acetyl-salicylic acid (ASA) and clopidrogel, vasodilating agents, e.g., nitrates, calcium channel blocking drugs, anti-proliferative agents, e.g., colchicine and alkylating agents, intercalating agents, growth modulating factors such as interleukins, transformation growth factor-beta and congeners of platelet derived growth factor, monoclonal antibodies directed against growth factors, anti-inflammatory agents, both steroidal and non-steroidal, and other agents that can modulate vessel tone, function, arteriosclerosis, and the healing response to vessel or organ injury post intervention. Antibiotics can also be included in combinations or coatings comprised by the invention. Moreover, a coating can be used to affect therapeutic delivery focally within the vessel wall. By incorporation of the active agent in a swellable polymer, the active agent will be released upon swelling of the polymer.

Medicaments which may be administered in conjunction with the methods described herein include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatories, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon. It will be clear to a person skilled in the art that, where appropriate, the medicaments may be used in the form of salts (e.g., as alkali metal or amine salts or as acid addition salts) or as esters (e.g., lower alkyl esters) or as solvates (e.g., hydrates) to optimize the activity and/or stability of the medicament.

Other exemplary therapeutic agents useful for a combination therapy include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Examples of therapeutic antibodies that can be combined with a subject compound include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

Further therapeutic agents that can be combined with a subject compound may be found in Goodman and Gilman's “The Pharmacological Basis of Therapeutics” Tenth Edition edited by Hardman, Limbird and Gilman or the Physician's Desk Reference, both of which are incorporated herein by reference in their entirety.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### EXAMPLES

### Example 1

### Antitumor Activity of Compound A in Breast Cancer Model

Objectives of this study: to determine the in vivo antitumor activity of compound A at various doses and schedules in the MDA-MB-361 human breast cancer xenografts after oral (PO) administration in female nude mice.

Compound A was prepared according to methods disclosed in WO 2011/022439 and WO 2013/071272 and stored at approximately 25° C., shielded from light. The control used in this study was 100% PEG400.

Low passage MDA-MB-361 cells were grown in Dulbecco's Modified Eagle's Medium (DMEM) media supplemented with 20% fetal bovine serum (FBS) and 1× penicillin/streptomycin (Invitrogen [Carlsbad, Calif., USA]) until approximately 80% confluency was reached. Prior to injection, the cells were detached with Trypsin (Invitrogen), washed twice with phosphate buffered saline (PBS), and resuspended in RPMI (Roswell Park Memorial Institute) media (Invitrogen,) without supplements. Cells were resuspended to a final concentration of 10.0×10⁷ cells/mL in DMEM media without supplements, with 50% Matrigel (BD Biosciences). MDA-MB-361 cells (10.0×10⁶ cells per animal in 0.1-mL injection volume) were implanted SC into the flank of female athymic nude mice (9 weeks at start of dosing).

Four-five week old female athymic nude mice were inoculated SC in flank (cell suspension in serum free DMEM and 50% Matrigel) with 10.0×10⁶ MDA-MB-361 cells. Tumor growth was monitored with vernier calipers. The mean TV (MTV) was calculated using the formula V=W²×L/2.

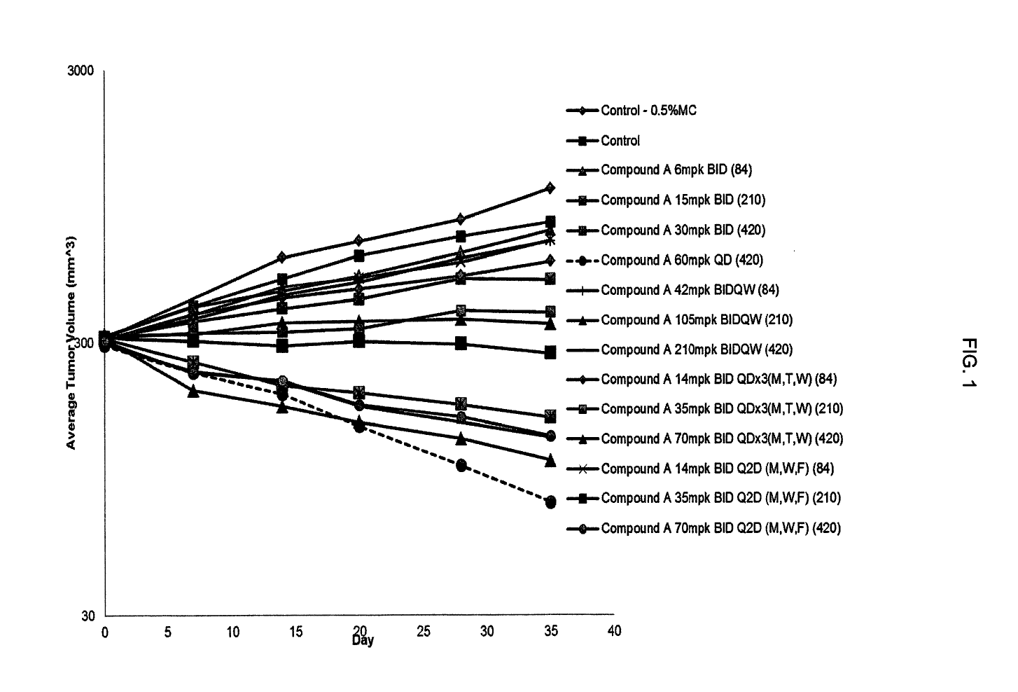

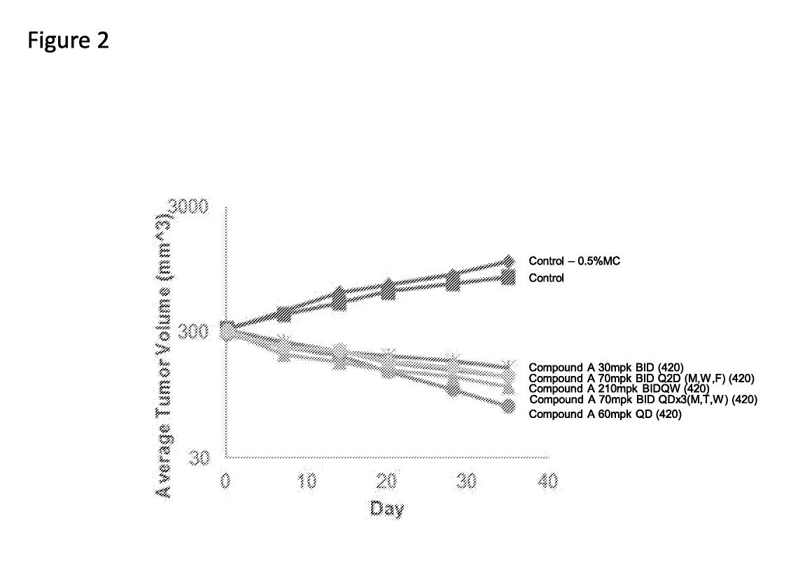

When tumors reached a mean tumor volume (MTV) of approximately 280 mm³, mice (n=5/group) were treated with vehicle (PEG400) or compound A (60, 70, 140, or 210 mg/kg) administered concurrently via oral gavage over a 28 day treatment period on the schedules indicated below in Table 2. The percentage of tumor growth inhibition (TGI) and percent body weight (BW) change were determined on Day 28. Statistical comparisons of tumor growth between the treatment and vehicle groups were conducted using a linear mixed effects regression analysis on the change in area under the tumor volume (TV)-versus—time curves (ΔAUC) values. All p values less than 0.05 were considered significant. The results of this study were provided in FIGS. 1, 2, and 3. FIG. 1 shows compound A's efficacy in various dosages and dosing schedules. FIG. 2 shows compound A's efficacy in various dosing schedules with the same drug amount per week and that similar efficacy can be obtained with the same amount of drug per week despite differences in dosing schedules. FIG. 3 shows the relationship between treatment efficacies (tumor growth inhibition, TGI) and exposure (AUC) between different dosing schedules, indicating that the efficacy of compound A as a single agent was AUC-driven. The results demonstrate that administration of compound A according to an intermittent regimen is as efficacious in reducing tumor size as daily administration, when the total amount of compound A administered is comparable in both the daily and intermittent schedules.

### Example 2

### Clinical Study with Compound A as the Single Agent

A phase 1 clinical study was initiated to evaluate the safety, tolerability, PK, pharmacodynamics; to determine the maximum tolerated dose (MTD) in each of 3 schedules described below; and to evaluate antitumor activity in patients with advanced solid malignancies.

### Eligibility

| | |
|---|---|
| Ages Eligible for Study: | 18 Years and older |
| Genders Eligible for Study: | Both |
| Accepts Healthy Volunteers: | No |

### Criteria

Inclusion Criteria
Subjects have had their PIK3CA gene mutation status assessed prior to enrolling into the study
Subjects must have documented disease progression prior to enrolling into the study
locally advanced or metastatic solid tumors with the exception of primary brain tumor, and have failed or are not eligible for standard of care therapy.
Age ≧18 years, including males and females;
ECOG performance status (PS) 0-1;
Adequate organ function;
Male subjects must be surgically sterile or must agree to use physician-approved contraception during the study and for 30 days following the last study drug administration;
Ability to swallow oral medications;
Ability to understand and willingness to sign informed consent prior to initiation of any study procedures;
For women of child-bearing potential, negative serum pregnancy test within 14 days prior to the first study drug administration and use of physician-approved method of birth control from 30 days prior to the first study drug administration to 30 days following the last study drug administration

Exclusion Criteria
Diagnosis of primary brain tumor; untreated brain metastasis or history of leptomeningeal disease or spinal cord compression;
Received prior cancer or other investigational therapy within 2 weeks prior to the first administration of study drug;
Have received a systemic corticosteroid within one week prior to the first administration of study drug;
Clinically significant cardiac disease;
Myocardial infarction or unstable angina within 6 months prior to the first administration of study drug;
Malabsorption;
Poorly controlled diabetes mellitus;
Pregnancy (positive serum or urine pregnancy test) or breast feeding;
Untreated brain metastasis or history of leptomeningeal disease or spinal cord compression;
Failed to recover from the reversible effects of prior anticancer therapies;
Have received a selective PI3K-alpha inhibitor
Other clinically significant co-morbidities, such as uncontrolled pulmonary disease, active central nervous system (CNS) disease, active infection, or any other condition that could compromise the subject's participation in the study
Known human immunodeficiency virus (HIV) infection
Have a secondary malignancy within the last 3 years prior to first dose of study drug, excluding treated non-melanoma skin cancer, carcinoma in situ, or locally-treated prostate cancer

35 patients were enrolled into the dose escalation phase of the study, which is designed to determine the MTD of compound A when given orally on continuous (daily) or intermittent schedules. See table 3 below. The MTD for the QD dosing schedule was determined to be 100 mg. The MTDs for the intermittent schedules (MTW and MWF) are expected to be higher than that of continuous (daily) regimen.

TABLE 3
Arms

Experimental: Arm A
Compound A administered once a day orally
Experimental: Arm B
Compound A administered orally intermittently, once every
other day (Monday, Wednesday, and Friday) each week
Experimental: Arm C
Compound A administered orally intermittently, once a day
for 3 consecutive days (Monday, Tuesday, and Wednesday)
each week

Other than MTD, the other primary outcome measures is the tolerability profile of compound A or the number of adverse events (AE) from the first dose of study drug through 30 days after the last dose of study drug. Plasma levels of compound A is one of the secondary outcome measures.

The frequency of AEs grade 3 or higher in this study is shown in table 4 below.

TABLE 4

| | QD (mg) | | | | | MWF (mg) | | | | | | | MTW (mg) | | | | |
|---|---|---|---|---|---|---|---|---|---|---|---|---|---|---|---|---|---|
| Preferred Term | 100 | 150 | 200 | 300 | Total | 200 | 300 | 400 | 600 | 900 | 1200 | Total | 200 | 400 | 600 | 900 | Total |
| n (%) | n = 6 | n = 6 | n = 8 | n = 4 | n = 24 | n = 3 | n = 3 | n = 3 | n = 3 | n = 6 | n = 2 | n = 20 | n = 3 | n = 3 | n = 4 | n = 6 | n = 16 |
| ALT increased | | | 1 | 2 | 3 (13) | | | | | 1 | | 1 (5) | | | | | |
| AST increased | | | | 2 | 2 (8) | | | | | 1 | | 1 (5) | | | | | |
| Transaminases | | 1 | 1 | | 2 (8) | | | | | | | | | | | | |
| incr'd | | | | | | | | | | | | | | | | | |
| Hyperglycaemia | | | | | | | | | | | | | | | 1 | 1 | 2 |
| Anaemia | | | | | | | | 1 | | | | 1 (5) | | | | | |
| Abdominal pain | | | 1 | | 1 (4) | | | | | | | | | | | | |
| Diarrhoea | | | | | | | | | | | | | | | 1 | | 1 (6) |
| Nausea | | | | | | | | | | | | | | | | 1 | 1 (6) |
| Chills | | | | | | | | | | | | | | 1 | | | 1 (6) |
| Drug-induced | | | 1 | | 1 (4) | | | | | | | | | | | | |
| liver injury | | | | | | | | | | | | | | | | | |
| Oxygen | | | | | | | | | | | | | | 1 | | | 1 (6) |
| saturation | | | | | | | | | | | | | | | | | |
| Decreased | | | | | | | | | | | | | | | | 1 | 1 (6) |
| appetite | | | | | | | | | | | | | | | | | |

### Example 3

### Antitumor Activity of Compound A in Combination with Fulvestrant in Human Breast Cancer Xenografts T47-D

Objectives of this study: to determine the antitumor activity of compound A dosed on intermittent schedule in combination with fulvestrant in T47D (PIK3CA mutant, ER/PR+) human breast cancer xenografts.

Compound A was prepared according to methods disclosed in WO 2011/022439 and WO 2013/071272, and formulated in 100% PEG400 and stored at approximately 25° C., shielded from light. The control used in this study was 100% PEG400+0.9% Saline. Fulvestrant was formulated in 0.9% saline.

Low passage T47-D cells were grown in in RPMI 1640 media supplemented with 10% fetal bovine serum (FBS). Prior to injection, the cells were detached with Trypsin (Invitrogen), washed twice with phosphate buffered saline (PBS), and resuspended in RPMI (Roswell Park Memorial Institute) media (Invitrogen,) without supplements. Cells were resuspended to a final concentration of 10.0×10⁷ cells/ml in DPBS and mixed with 50% Matrigel (BD Biosciences) at 1 to 1 volume. T47-D cells (10.0×10⁶ cells/animal in 02-mL injection volume) were implanted S.C. into the right flank of NOD-SCID mice (Harlain Laboratories). 17β-ESTRADIOL, 0.36 mg 90-day release, tablets (Innovative Research of America) were implanted into mice 3 days prior to tumor cell implant.

Four-six week old female NOD-SCID mice were inoculated SC in flank (cell suspension in DPBS and 50% Matrigel) with 10.0×10⁶ T47-D cells. Tumor growth was monitored with vernier calipers. The mean TV (MTV) was calculated using the formula V=W2×L/2.

When tumors reached a mean tumor volume (MTV) of approximately 250 mm³, mice (n=8/group) were treated with vehicle (100% PEG400+0.9% saline, QDx21), compound A at 70 mg/kg (QDx3 schedule via oral gavage, PO), 100 mg/kg fulverstrant (QW schedule, dosed subcutaneously SC), and combination of compound A with fulvestrant administered concurrently over a 21 day treatment period, see study details in Table 5. The percentage of tumor growth inhibition (TGI) and percent body weight (BW) change were determined on Day 21 and provided in Table 6. Statistical comparisons of tumor growth between the treatment and vehicle groups were conducted using a linear mixed effects regression analysis on the change in area under the tumor volume (TV)-versus—time curves (ΔAUC) values. All p values less than 0.05 were considered significant.

A combination score calculation was used to address the question of whether the effects of the combination treatments were synergistic, additive, subadditive, or antagonistic relative to the individual treatments. The effect was considered synergistic if the synergy score was less than 0, and additive if the synergy score wasn't statistically different from 0. If the synergy score was greater than 0, but the mean AUC for the combination was lower than the lowest mean AUC among the two single agent treatments, then the combination was subadditive. If the synergy score was greater than zero, and the mean AUC for the combination was greater than the mean AUC for at least one of the single agent treatments, then the combination was antagonistic.

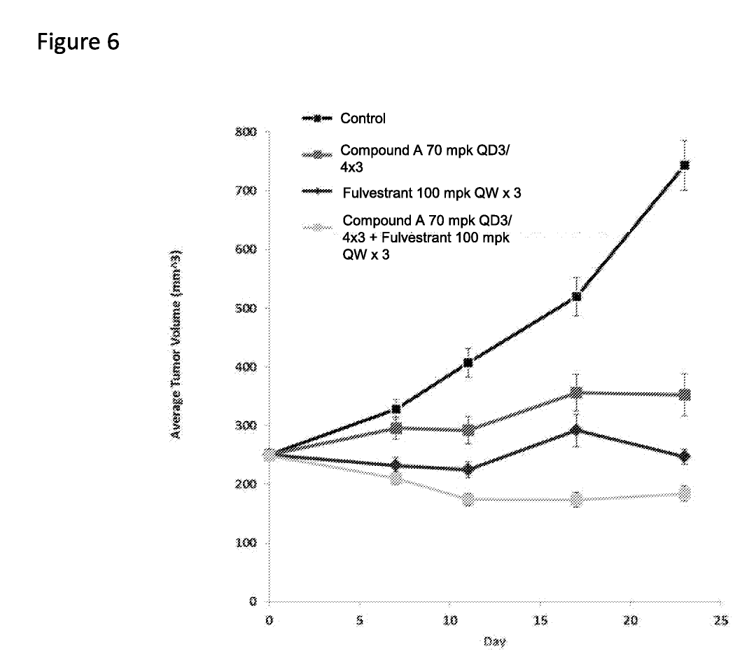

Results of this study were provided in FIG. 6. FIG. 6 shows efficacy of compound A as a single agent, efficacy of fulvestrant as a single agent and the combination efficacy of both drugs dosed concurrently over a 21 day treatment period. The results demonstrate that administration of compound A is efficacious (TGI is 52.6%) in reducing tumor size of T47-D xenografts when compound A is administered on intermittent schedule. Combination treatment shows that addition of compound A to fulvestrant may provide added tumor growth inhibition benefit in ER/PR+models. A combination score analysis demonstrates that in this study the combination of compound A with fulvestrant was additive.

TABLE 5

| | Number | | | | |
|---|---|---|---|---|---|
| Group | of | | Dose | | |
| #: | Mice | Treatment | (mg/kg) | Schedule | Route |
| 1 | 8 | 100% PEG400 | n/a | QD1-21 | PO |
| | | 0.9% saline | n/a | QD1-21 | SC |
| 2 | 8 | Compound A | 70 | QD3/4 x3 | PO |
| 3 | 8 | Fulvestrant | 100 | QW x3 | SC |
| 4 | 8 | Fulvestrant | 100 | QW x3 | SC |
| | | Compound A | 70 | QD3/4 x3 | PO |
| 5 | 8 | Fulvestrant | 100 | QW x3 | SC |

TABLE 6

| | | Method of | | | |
|---|---|---|---|---|---|
| Treatment | Dose ^{a} | Administration/ | | | |
| Group | (mg/kg) | Frequency | Endpoints | Noteworthy Findings | |
| PEG400 | NA | PO/QD x 21 | TGI ^{a} | N/A | |
| Saline | NA | Days 1-21 | | | |
| (0.9%) | | SC/QW x 3 | Maximum Mean % BWL ^{b} | 2.1% | (Day 17) |
| Compound A | 70 | PO/QD x 3/wk x 3 | TGI ^{a} | 52.60% | |
| | | Days 1-3, 8-10, 15-17 | ΔAUC ^{c} | 61.2 | (p < 0.001) |
| Fulvestrant | 100 | SC/QW x 3 | Maximum Mean % BWL ^{b} | 0% | |
| | | Days 1, 8, 15 | TGI ^{a} | 66.70% | |
| | | | ΔAUC ^{c} | 108.4 | (p < 0.001) |
| | | | Maximum Mean % BWL ^{b} | 1% | (Day 7) |
| Compound A + | 70 | PO/QD x 3/wk x 3 | TGI ^{a} | 72.50% | |
| Fulvestrant | 100 | Days 1-3, 8-10, 15-17 | ΔAUC ^{c} | 165.8 | (p < 0.001) |

| SC/QW x 3 | Synergy analysis ^{d} | Additive | |
|---|---|---|---|
| Days 1, 8, 15 | Maximum Mean % BWL ^{b} | 4% | (Day 7) |

^{a} TGI values were calculated on Day 21 post treatment initiation
^{b} Maximum mean percent BWL between Day 0 to Day 21
^{c} ΔAUC = Statistical analysis was performed with a linear mixed effects regression model. A p value of < 0.05 was considered significant.
^{d} Synergistic analysis: p > 0.05 = additive; p < 0.05 and score < 0 = synergistic; p < 0.05, score > 0, and the combination growth rate is lower than both the single agent growth rates = subadditive; p < 0.05, score > 0, and the combination growth rate is higher than at least one of the single agent growth rates = antagonistic. P values < 0.05 were considered statistically significant.

1. A pharmaceutical regimen for treating cancer comprising administering intermittently to a subject in need thereof a therapeutically effective amount of a PI3Kα inhibitor for at least one week, wherein the PI3Kα inhibitor is (6-(2-aminobenzo[d]oxazol-5-yl)imidazo[1,2-a]pyridin-3-yl)(morpholino)methanone, wherein the therapeutically effective amount is from about 600 mg to about 3000 mg weekly.

2. (canceled)

3. The pharmaceutical regimen of claim 1 wherein the therapeutically effective amount is from about 600 mg to about 900 mg.

4. The pharmaceutical regimen of claim 1 wherein the therapeutically effective amount is from about 900 mg to about 1200 mg.

5. The pharmaceutical regimen of claim 1 wherein the therapeutically effective amount is from about 1200 mg to about 1800 mg.

6. (canceled)

7. The pharmaceutical regimen of claim 1 wherein the therapeutically effective amount is about 900 mg.

8.-12. (canceled)

13. The pharmaceutical regimen of claim 1, comprising at least one 7-day cycle in which the PI3Kα inhibitor is administered for at least one day followed by an intermission in which the PI3Kα inhibitor is not administered for at least one day.

14. The pharmaceutical regimen of claim 1, wherein the PI3Kα inhibitor is administered for 2, 3, 4, 5, 6, or 7 consecutive days, followed by an intermission in which the PI3Kα inhibitor is not administered for at least 1, 2, 3, 4, 5, or 6 days.

15. The pharmaceutical regimen of claim 1, wherein the PI3Kα inhibitor is administered three days a week.

16. The pharmaceutical regimen of claim 1, wherein the PI3Kα inhibitor is administered on consecutive days during the week and followed by an intermission.

17. The pharmaceutical regimen of claim 16 comprising at least one 7-day cycle in which the PI3Kα inhibitor is administered for 3 consecutive days followed by an intermission of 4 consecutive days.

18. The pharmaceutical regimen of claim 1, wherein the PI3Kα inhibitor is administered on alternate days during the week and followed by an intermission.

19. The pharmaceutical regimen of claim 18 comprising administering the PI3Kα inhibitor at least 3 times on alternative days within a 7-day cycle.

20. The pharmaceutical regimen of claim 1, wherein the PI3Kα inhibitor is administered once a day (QD) in each of the days that the PI3Kα inhibitor is administered to the human subject.

21. The pharmaceutical regimen of claim 1, wherein the PI3Kα inhibitor is administered twice a day (BID) in each of the days that the PI3Kα inhibitor is administered.

22. The pharmaceutical regimen of claim 1, wherein said regimen achieves an area under the curve (AUC) greater than 45 μg*h/mL, 48.5 μg*h/mL, or 50 μg*h/mL in the subject over a dosing day; or 100 μg*h/mL, 150 μg*h/mL, or 200 μg*h/mL in the subject over a dosing week.

23.-30. (canceled)

31. The pharmaceutical regimen of claim 1, wherein an additional therapeutic agent is administered to the subject.

32. The pharmaceutical regimen of claim 31, wherein the additional therapeutic agent is an anticancer agent.

33. The pharmaceutical regimen of claim 31, wherein the additional therapeutic agent is selected from one or more of paclitaxel, fulvestrant, exemestane, gemcitabine, erlotinib, gefitinib, afatinib, nintedanib, dacomitinib, bevacizumab, pemetrexed, motesanib, crizotinib, ipilimumab, ramucirumab, custirsen, and onartuzumab.

34. The pharmaceutical regimen of claim 33, wherein the additional therapeutic agent is fulvestrant.

35. The pharmaceutical regimen of claim 1, wherein the cancer is selected from the group consisting of non-small cell lung cancer, small cell lung cancer, head and neck squamous cell carcinoma, pancreatic cancer, breast cancer, ovarian cancer, renal cell carcinoma, prostate cancer, neuroendocrine cancer, gastric cancer, bladder cancer, colon cancer and endometrial cancer.

36.-65. (canceled)
